(19) United States
(12) Patent Application Publication
(10) Pub. No.: US 2019/0085031 A1
(43) Pub. Date: Mar. 21, 2019

(54) PROTEIN VARIANTS FOR USE AS LIPID BILAYER-INTEGRATED NANOPORE, AND METHODS THEREOF

(71) Applicant: UNIVERSITY OF KENTUCKY RESEARCH FOUNDATION, Lexington, KY (US)

(72) Inventors: Peixuan Guo, Columbus, OH (US); Shaoying Wang, Columbus, OH (US)

(21) Appl. No.: 15/760,670

(22) PCT Filed: Sep. 16, 2016
(86) PCT No.: PCT/US16/52158

§ 371 Date: Mar. 16, 2018

Related U.S. Application Data
(60) Provisional application No. 62/220,545, filed on Sep. 18, 2015.

(51) Int. Cl. C07K 14/005; G01N 33/68; C12Q 1/6869; G01N 33/487

(52) U.S. Cl. CPC C07K 14/005; G01N 33/6872; C12Q 1/6869; G01N 33/48721; C07K 2319/20; C12N 2795/10122; C12N 2795/10222; C07K 2319/22; C07K 2319/21; C07K 2319/10

(57) ABSTRACT

The presently-disclosed subject matter relates to an engineered T3 or T4 viral DNA-packaging motor connector protein that can be incorporated into a lipid membrane to form an electroconductive aperture, and which can be provided for other uses described herein.

### RELATED APPLICATIONS

This application claims the benefit of U.S. Patent Application No. 62/220,545, filed Sep. 18, 2015, the contents of which are incorporated by reference in their entirety.

### GOVERNMENT INTEREST

This presently disclosed subject matter was made with Government support under Grant No. R01EB012135 awarded by the National Institutes of Health. The Government has certain rights in the invention.

### TECHNICAL FIELD

The presently-disclosed subject matter relates to an engineered T3 and/or T4 viral DNA-packaging motor connector protein that can be incorporated into a lipid membrane to form an electroconductive aperture, and which can be provided for other uses described herein.

### INTRODUCTION

DNA translocation motors are ubiquitous in all living systems (1-6). During replication, the genome of double stranded DNA (dsDNA) viruses was packaged into a preformed protein referred as prohead to a density similar to that of crystalline DNA (7). This process is entropically unfavorable and requires a powerful packaging motor to accomplish the task. The component of packaging motors in many dsDNA bacteriophages and herpes viruses includes a protein channel called connector or portal vertex. It is located between the capsid shell and the ATPase ring. pRNA is a unique component in bacteriophage phi29 required for genomic DNA packaging (8, 9). Structural studies have shown that connectors from herpes virus and different tailed bacteriophages, such as phi29, SPP1, T4, and T3, share similar cone-shaped dodecamer structure (FIG. 1), even though their primary sequences do not display homology. The connector protein plays a critical role in genome packaging and ejection. During viral assembly, the connector serves as a docking point for motor ATPase and a conduit for dsDNA transport. After DNA packaging, the connector then serves as a binding site for tail components to complete virion assembly. When bacteriophages start to infect, the DNA is ejected through the coaxial connector and tail channel into the host cell.

Recent studies showed that the DNA translocase of bacteriophage phi29 uses a “Revolving through one-way valve” (10-12) rather than rotation (13) to package DNA, and T4 uses a “Torsional compression” mechanism (14, 15). Since the channels act like a one-way valve, an obvious question is how dsDNA is ejected during infection if the channel is a one-way inward valve. Previous studies have revealed an unexplained phenomenon that, after DNase digestion, the packaging intermediates or incompletely packaged DNA always showed three major bands in phi29 and T3 (16, 17). Earlier studies have demonstrated that the connector exercises conformation changes during DNA packaging and ejection processes. For example, one of the studies showed that phi29 connector conformation change was induced by DNA, pRNA or divalent metal ions assayed by circular dichroism and quenching of intrinsic tryptophan fluorescence (18, 19). Cryo-EM has also revealed conformational changes of free in vitro connector and the connector in the infectious virion (20). However, none of these studies to date has shown the conformation changes at the single molecule level.

Nanopore technology is an emerging area with the potential for versatile applications, including sensing small molecules, macromolecules, molecular binding, protein folding and DNA sequencing (21-28). The reengineered phi29 gp10 connector has been inserted into a lipid bilayer showing highly robust properties that can withstand a wide range of solution conditions, including pH 2-12, and ionic strengths of 0.1-3M NaCl or KCl (29, 30). The insertion of the connector channel into the lipid membrane results in homogenous step size increases in current and the channel exhibits equal conductance under both positive and negative voltage (29). By introducing appropriate probe at either the interior, or the terminal ends of the channel, single chemical or single antibody can be detected at ultra-low concentrations based on the current signature (31, 32). The channel allows dsDNA translocation (10, 11, 29, 33-36) and is able to discriminate ss-DNA and RNA with appropriate modification (34). Furthermore, that phi29 connector channel displays a one-way traffic property for dsDNA translocation with a valve mechanism in DNA packaging (10, 11) and voltage-induced channel gating (35). A finding of a conformation change of the channel that is common to DNA translocases of bacteriophage T3, T4, SPP1, and Phi29 supports the observation that the one way inbound channel was transformed into an outbound channel during the DNA ejection process.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

In some embodiments, the presently disclosed subject matter provides an engineered nucleic acid molecule encoding a T3 or T4 viral connector polypeptide variant. In some embodiments, the polypeptide comprises an amino acid sequence having at least 95% sequence identity to SEQ ID NO: 2. In some embodiments, the polypeptide comprises an amino acid sequence having at least 99% sequence identity to SEQ ID NO: 2. In some embodiments, the polypeptide comprises the amino acid sequence of SEQ ID NO: 2. In some embodiments, the nucleic acid sequence comprises the sequence of SEQ ID NO: 1. In some embodiments, the polypeptide comprises an amino acid sequence having at least 95% sequence identity to SEQ ID NO: 4. In some embodiments, the polypeptide comprises an amino acid sequence having at least 99% sequence identity to SEQ ID NO: 4. In some embodiments, the polypeptide comprises the amino acid sequence of SEQ ID NO: 4. In some embodiments, the nucleic acid sequence comprises the sequence of SEQ ID NO: 3.

Further provided, in some embodiments of the presently provided subject matter, is an engineered T3 or T4 viral connector polypeptide variant. In some embodiments, the polypeptide variant comprises an amino acid sequence having at least 95% sequence identity to the sequence of SEQ ID NO: 2 or SEQ ID NO: 4. In some embodiments, the viral connector polypeptide variant comprises an amino acid sequence having at least 99% sequence identity to the sequence of SEQ ID NO: 2 or SEQ ID NO: 4. In some embodiments, the viral connector polypeptide variant comprises an amino acid sequence having the sequence of SEQ ID NO: 2 or SEQ ID NO: 4. In some embodiments, the viral connector polypeptide variant comprises the sequence of SEQ ID NO. 2 or SEQ ID NO. 4.

Yet in some embodiments of the presently disclosed subject matter, an artificial conductive channel-containing voltage-gated membrane complex is provided. In some embodiments, the membrane comprises a membrane layer and an isolated DNA-packaging motor connector protein that is incorporated into the membrane layer to form an aperture through which conductance can occur when an electrical potential is applied across the membrane. In some embodiments, the DNA-packaging motor connector protein comprises a homododecamer of viral DNA-packaging motor connecting protein polypeptide subunits, In some embodiments, the subunits comprises an amino acid sequence having at least 95% identity to SEQ ID NO: 2 or SEQ ID NO: 4. In some embodiments, the viral DNA-packaging motor connector protein polypeptide subunits comprises an amino acid sequence having at least 99% identity to SEQ ID NO: 2 or SEQ ID NO: 4. In some embodiments, the viral DNA-packaging motor connector protein polypeptide subunits comprises sequence as set forth in SEQ ID NO: 2 or SEQ ID NO: 4.

The membrane of claim 9, wherein the viral DNA-packaging motor connector protein polypeptide subunits is encoded by a nucleic acid molecule comprising SEQ ID NO. 1 or SEQ ID NO. 3. In some embodiments, the subunit further comprises an affinity/alignment domain. In some embodiments, the affinity/alignment domain comprises a polypeptide of (i) a Strep-11 tag sequence WSHPQRFEK; (ii) a polyhistidine polypeptide tag of 3, 4, 5, 6, 7, 8, 9, 10, 11 or 12 contiguous histidine residues; (iii) a polyarginine polypeptide of 3, 4, 5, 6, 7, 8, 9, 10, 11 or 12 contiguous arginine residues; (iv) an HIV Tat polypeptide of sequence YGRKKRRQRR, and (v) a peptide tag of sequence DRATPY. In some embodiments, the membrane translocates double-stranded DNA through the aperture when the electrical potential is applied. In some embodiments, the conductance occurs in the conductive channel-containing membrane with voltage-gating when electrical potential is applied. In some embodiments, the applied electrical potential is greater than about 100 mV. In some embodiments, the electrical potential is greater than about 125 mV, greater than about 150 mV, greater than about 175 mV, greater than about 200 mV, greater than about 225 mV, greater than about 250 mV, greater than about 275 mV, greater than about 300 mV. In some embodiments, the applied electrical potential is less than about −100 mV. In some embodiments, the applied electrical potential is less than about −125 mV, less than about −150 mV, less than about −175 mV, less than about −200 mV, less than about −225 mV, less than about −250 mV, less than about −275 mV, less than about −300 mV. In some embodiments, the membrane layer comprises a lipid layer. In some embodiments, the lipid layer comprises amphipathic lipids. Non-limiting examples of the lipid layer include planar membrane layer and a liposome. In some embodiments, the amphipathic lipids comprise phospholipids and the lipid layer comprises a lipid bilayer. In some embodiments, the liposome includes but not limited to a multilamellar liposome and a unilamellar liposome. In some embodiments, the incorporated viral DNA-packaging motor connector protein is mobile in the membrane layer.

In some embodiments, the presently disclosed subject matter provides a method of sensing a molecule using a conductive channel-containing membrane as disclosed herein. The method comprises contacting the molecule with a conductive channel-containing membrane which comprises a membrane layer and incorporated therein one or a plurality of isolated viral DNA-packaging motor connector proteins, applying an electrical potential, and detecting electrical current change, wherein the current change is a discrete a 3-step change. In some embodiments, the discrete 3-step current change is about 33%, about 66%, and 99% reduction in each step. In some embodiments, the electrical potential is greater than about 100 mV. In some embodiments, the electrical potential is less than about −100 mV. In some embodiments, the molecule is a polypeptide. In some embodiments, the molecule is a nucleic acid molecule. In some embodiments, the nucleic acid molecule is a double-stranded nucleic acid molecule. In some embodiments, the presently disclosed subject matter further provides a method of DNA sequencing using a conductive channel-containing membrane as disclosed herein.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are used, and the accompanying drawings. The drawings were originally published in color, incorporated by reference in their entireties (Wang, S., et al., Three-step channel conformational changes common to DNA packaging motors of bacterial viruses T3, T4, SPP1, and Phi29, Virology. 2016 May 12. pii: S0042-6822(16)30073-3. doi: 10.1016/j.virol.2016.04.015). The black and white drawings of the instant application correspond to the color ones published.

FIG. 1 illustrates structures T4 and T3 connector channels. A schematic is drawn, since crystal structures are not available for T3, and T4.

FIG. 2 includes image showing coomasie-blue stained 10% SDS-PAGE showing the molecular weight differences of single subunit of T4 (60 kDa) and T3 (59 kDa) connector channels.

FIG. 3A shows representative current traces showing insertion of T4 connector channels into planar lipid membrane. Applied voltage: 50 mV; Conducting buffer: 1 M KCl, 5 mM HEPES, pH 7.8.

FIG. 3B shows representative current traces showing insertion of T3 connector channels into planar lipid membrane. Applied voltage: 50 mV; Conducting buffer: 1 M KCl, 5 mM HEPES, pH 7.8.

FIG. 4A shows histogram data showing the conductance distribution of T4 connector channels. Applied voltage: 50 mV; Conducting buffer: 1 M KCl, 5 mM HEPES, pH 7.8. The conductance value is reported as mean±standard deviation from three independent experiments.

FIG. 4B shows histogram data showing the conductance distribution of T3 connector channels. Applied voltage: 50 mV; Conducting buffer: 1 M KCl, 5 mM HEPES, pH 7.8. The conductance value is reported as mean±standard deviation from three independent experiments.

FIG. 5 shows Current-Voltage trace under a ramping potential (−50 mV to 50 mV; 2.2 my/s) for T4 (one channel) and T3 (three channels) connectors. Conducting buffer: 1 M KCl, 5 mM HEPES, pH 7.8.

FIG. 6 shows three step gating associated with conformational changes of T4 and T3 connector channel under positive trans-membrane voltages.

FIG. 7 shows three steps gating associated with conformational changes of T4 and T3 connector channels under negative trans-membrane voltage.

FIG. 8A shows top view, side view and single subunit of Phi29 portal channel structure.

FIG. 8B shows top view, side view and single subunit of SPP1portal channel structure.

FIG. 8C shows top view, side view and single subunit of T4 portal channel structure.

FIG. 9A sets forth representative current traces showing insertion of phi29 portal channel into planar lipid membrane.

FIG. 9B sets forth representative current traces showing insertion of SPP1 portal channel into planar lipid membrane.

FIG. 9C sets forth representative current traces showing insertion of T4 portal channel into planar lipid membrane.

FIG. 9D sets forth representative current traces showing insertion of T3 portal channels into planar lipid membrane.

FIG. 9E sets forth a histogram showing the conductance distribution of phi29 portal channels.

FIG. 9F sets forth a histogram showing the conductance distribution of SPP1 portal channels.

FIG. 9G sets forth a histogram showing the conductance distribution of T4 portal channels.

FIG. 9H sets forth a histogram showing the conductance distribution of T3 portal channels.

FIG. 9I shows Current-Voltage trace under a ramping potential (−50 mV to +50 mV; 2.2 mV/s) for phi29 (single channel) portal.

FIG. 9J shows Current-Voltage trace under a ramping potential (−50 mV to +50 mV; 2.2 mV/s) for SPPI (two channel) portal.

FIG. 9K shows Current-Voltage trace under a ramping potential (−50 mV to +50 mV; 2.2 mV/s) for T4 (one channel) portal.

FIG. 9L shows Current-Voltage trace under a ramping potential (−50 mV to +50 mV; 2.2 mV/s) for T3 (three channels) portal.

FIG. 10A shows three step gating associated with conformational changes of phi29 portal channel under positive trans-membrane voltages.

FIG. 10B shows three step gating associated with conformational changes of SPP1 portal channel under positive trans-membrane voltages.

FIG. 10C shows three step gating associated with conformational changes of T4 portal channel under positive trans-membrane voltages.

FIG. 10D shows three step gating associated with conformational changes of T3 portal channel under positive trans-membrane voltages.

FIG. 10E shows three step gating associated with conformational changes of phi29 portal channel under negative trans-membrane voltages.

FIG. 10F shows three step gating associated with conformational changes of SPP1 portal channel under negative trans-membrane voltages.

FIG. 10G shows three step gating associated with conformational changes of T4 portal channel under negative trans-membrane voltages.

FIG. 10H shows three step gating associated with conformational changes of T3 portal channel under negative trans-membrane voltages.

FIG. 11 is a Coomasie-blue stained 10% SDS-PAGE showing the molecular weight differences of single subunit of phi29 (36 kDa), SPP1 (56 kDa), T4 (60 kDa) and T3 (59 kDa) portal channels.

FIG. 12 shows a single channel insertion of T4 gp20 connector channel.

FIG. 13 shows multiple insertion of T4 gp-20 connector channel.

FIG. 14 shows TAT peptide translocation for the T4 connector.

FIG. 15 shows T4 connector peptide translocation.

FIG. 16 shows translocation of peptide through T4 gp20.

FIG. 17 shows peptide translocation through T3 connector.

FIG. 18 shows blockade of peptide translocation through T3 connector.

FIG. 19 shows one way traffic of peptide translocation through T3 connector.

### BRIEF DESCRIPTION OF THE SEQUENCE LISTING

SEQ ID NO: 1 is a nucleic acid sequence of the T3 mutant connector sequence as used in the presently disclosed subject matter.

SEQ ID NO: 2 is an amino acid sequence of T3 mutant connector sequence as used in the presently disclosed subject matter.

SEQ ID NO: 3 is a nucleic acid sequence of the T4 gp-20 mutant connector sequence as used in the presently disclosed subject matter.

SEQ ID NO: 4 is an amino acid sequence of T4 gp-20 mutant connector sequence as used in the presently disclosed subject matter.

### DESCRIPTION OF EXEMPLARY EMBODIMENTS

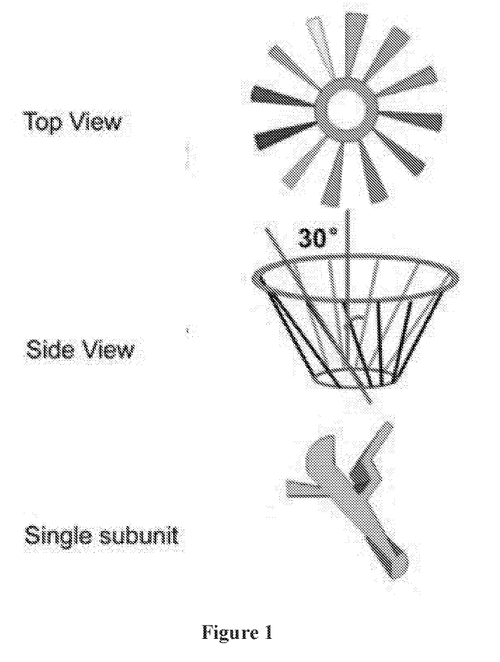

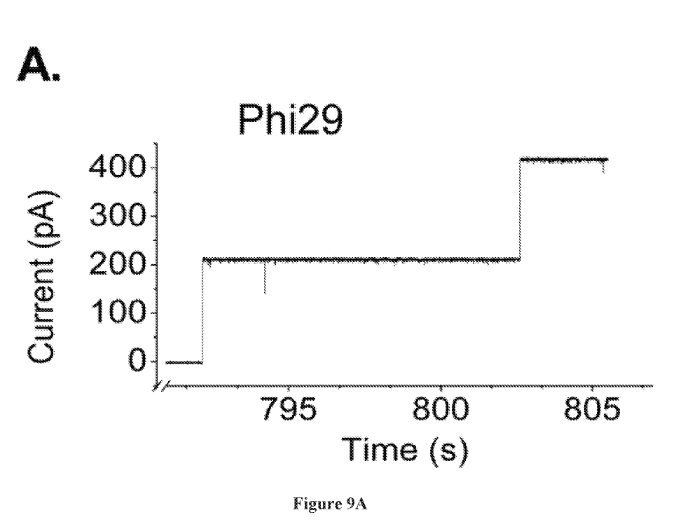

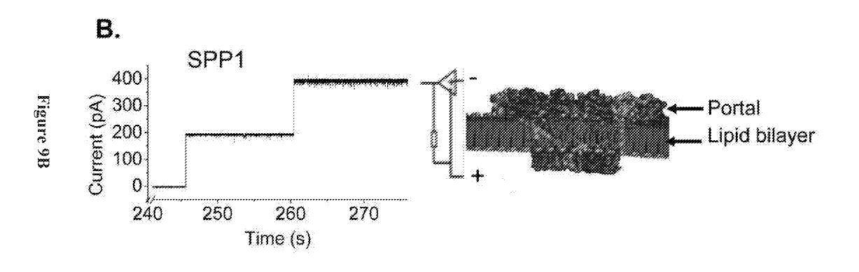

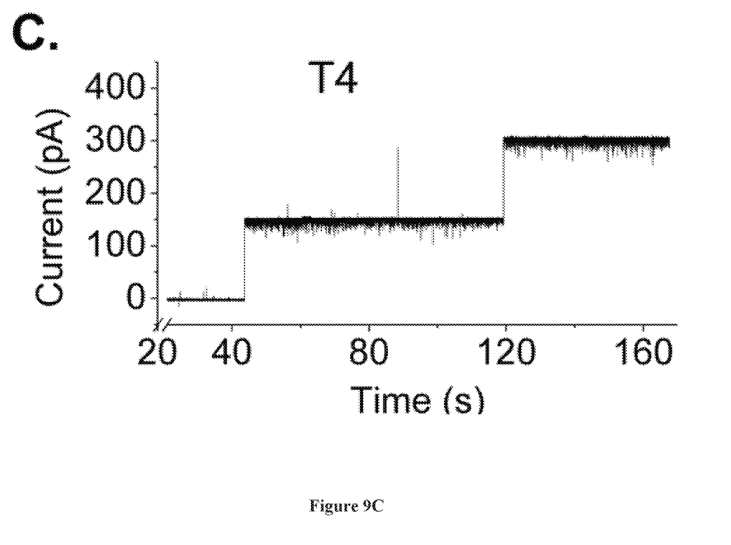

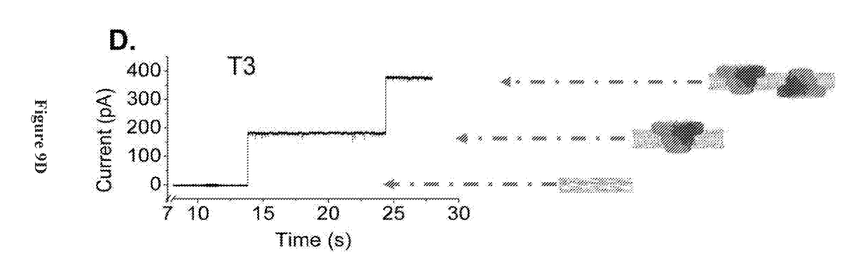

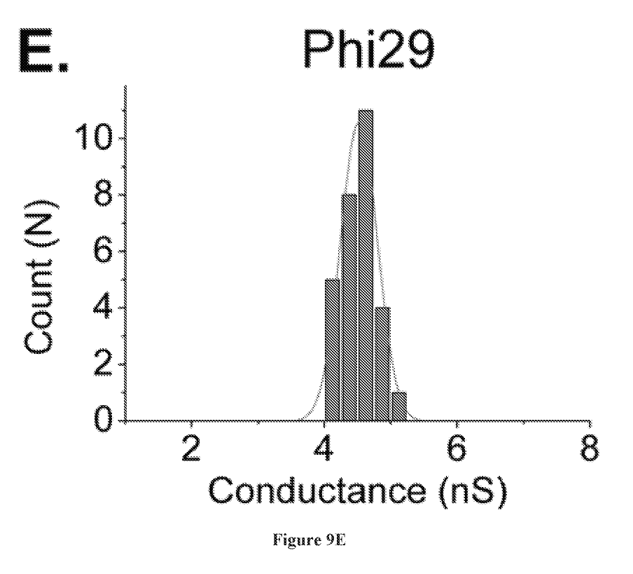

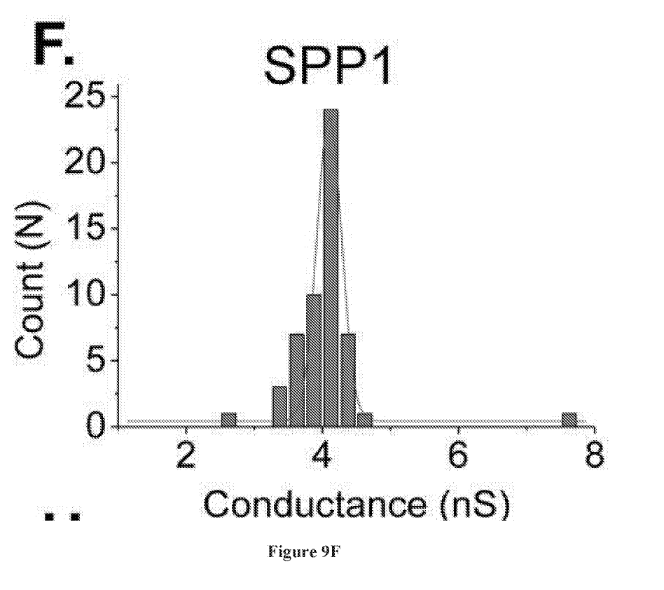

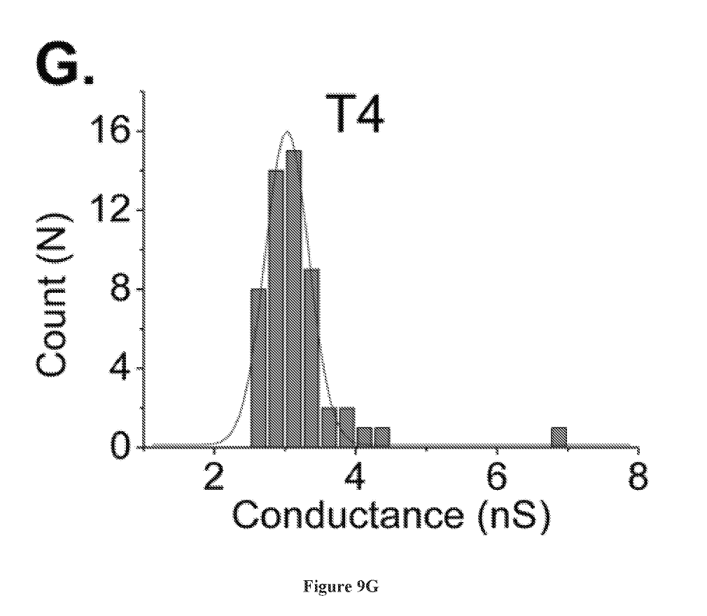

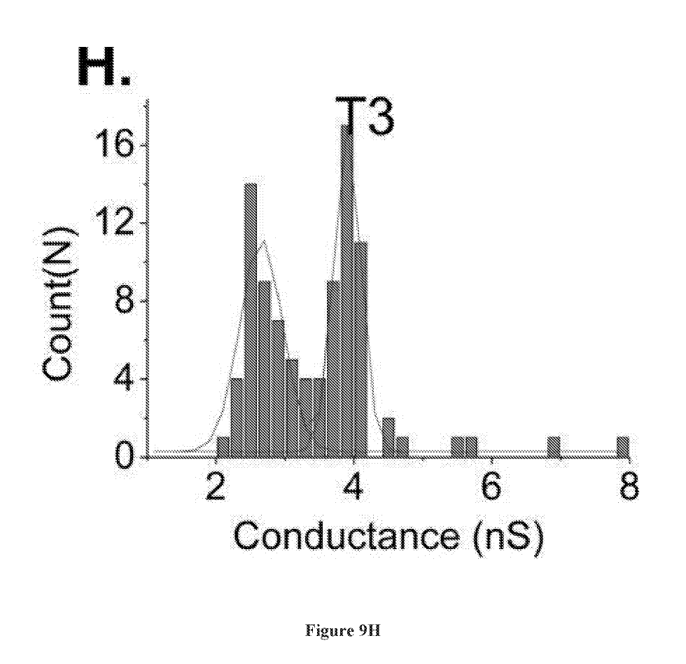

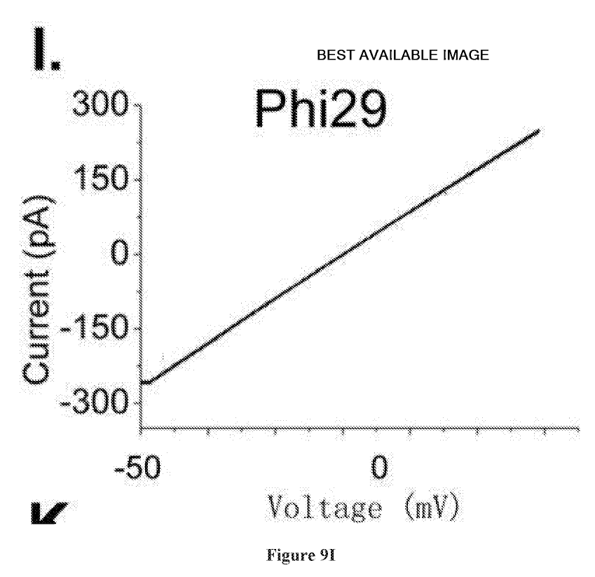

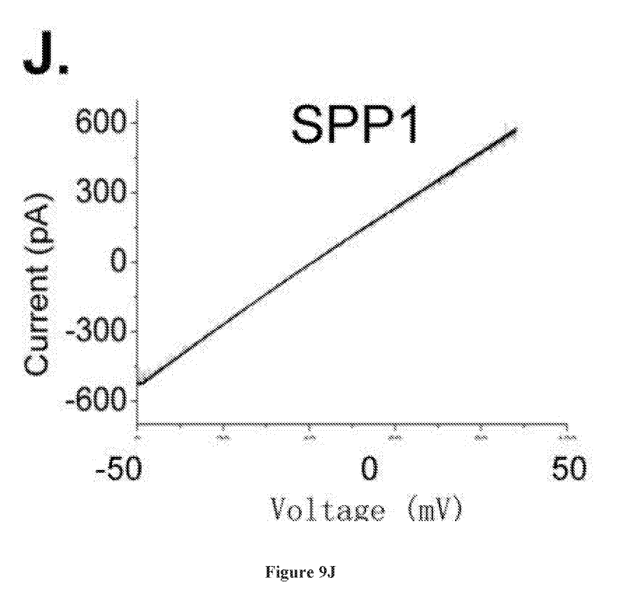

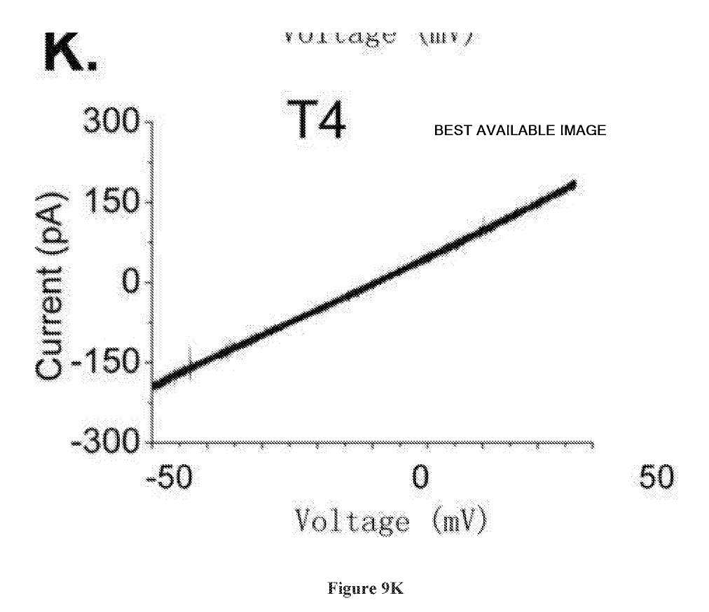

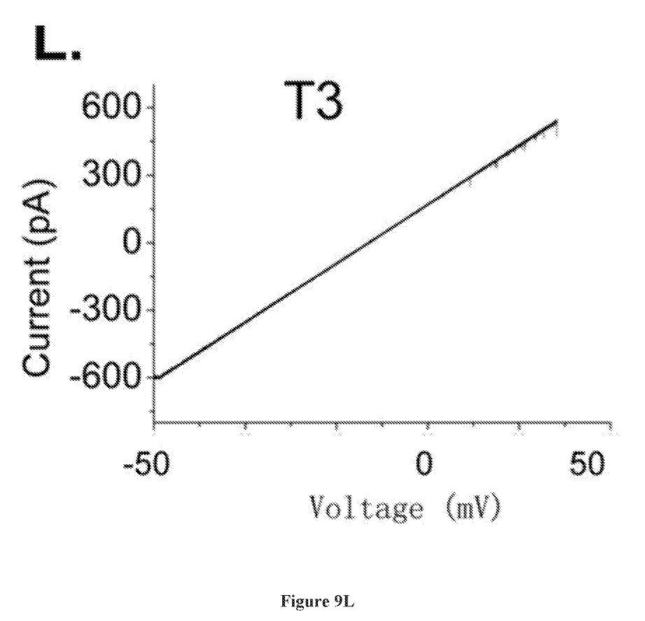

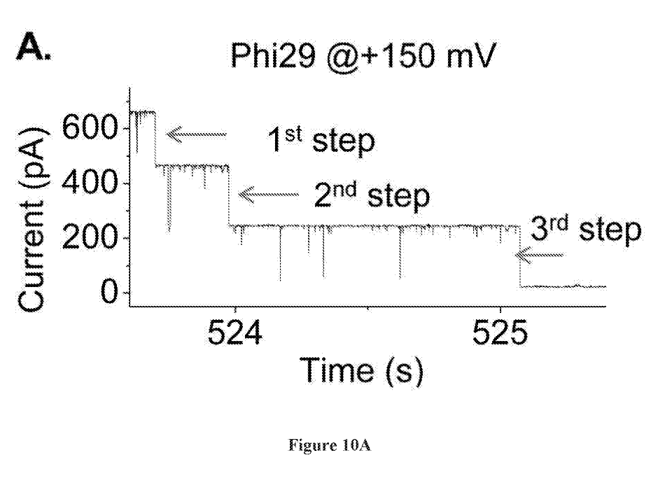

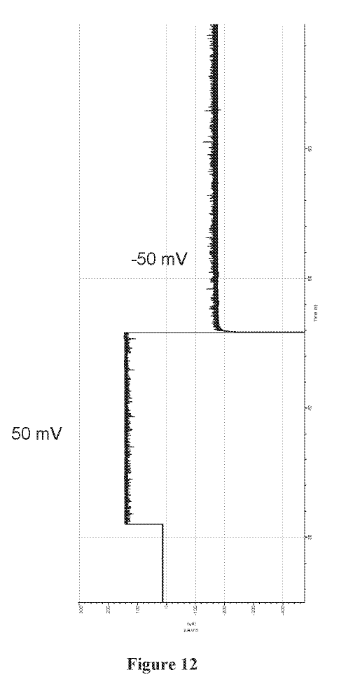

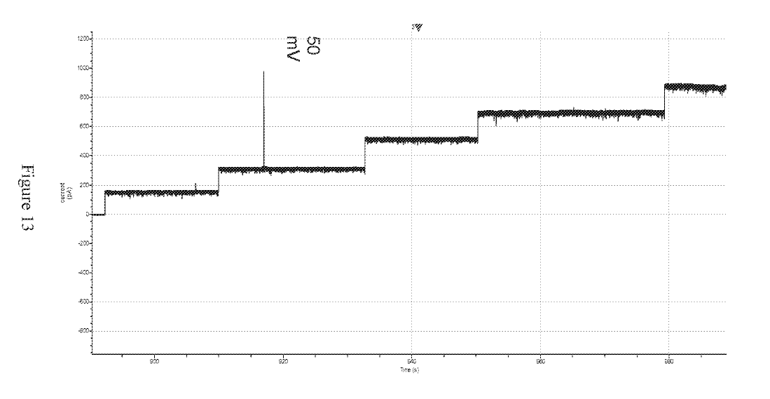

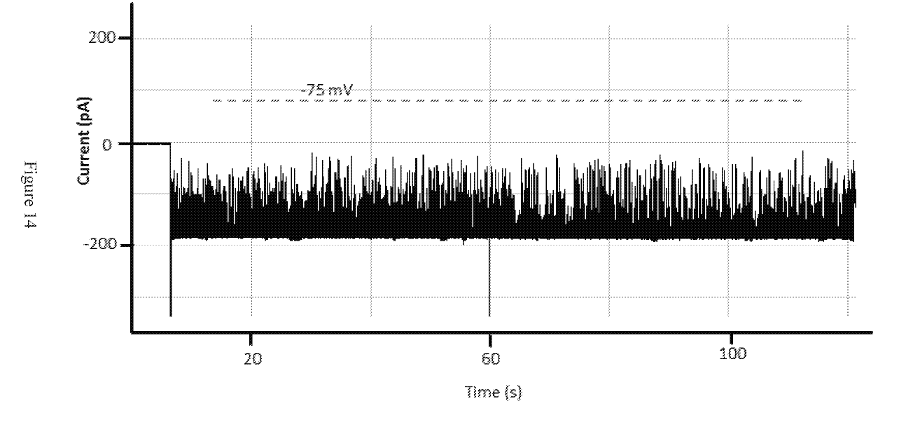

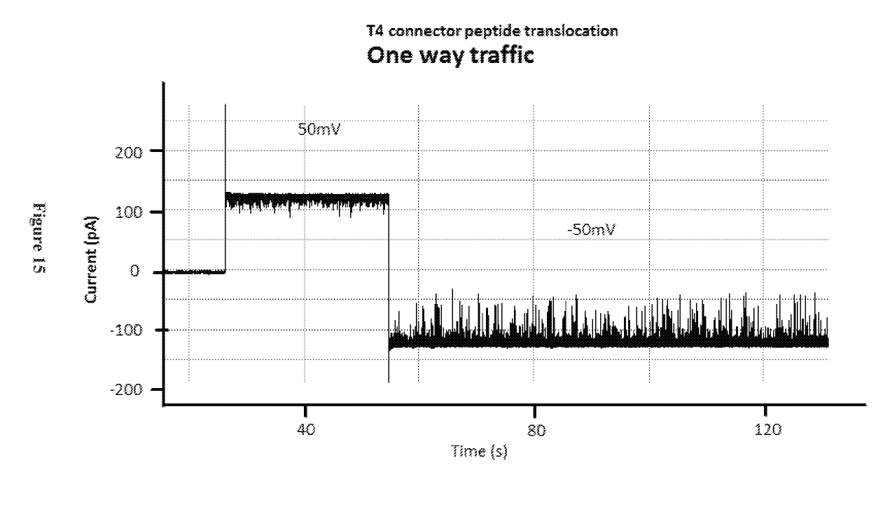

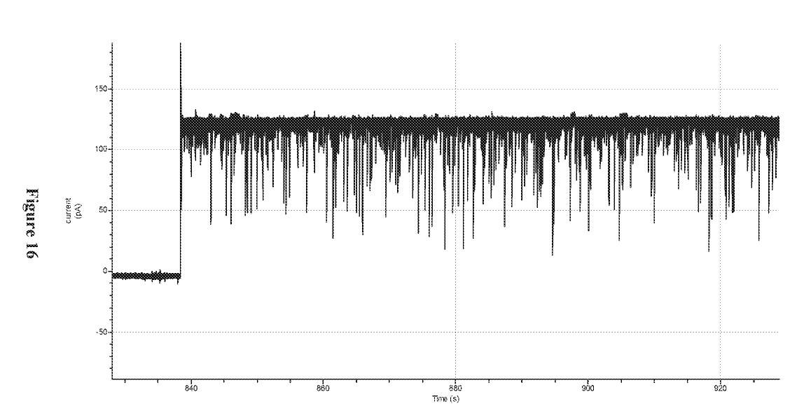

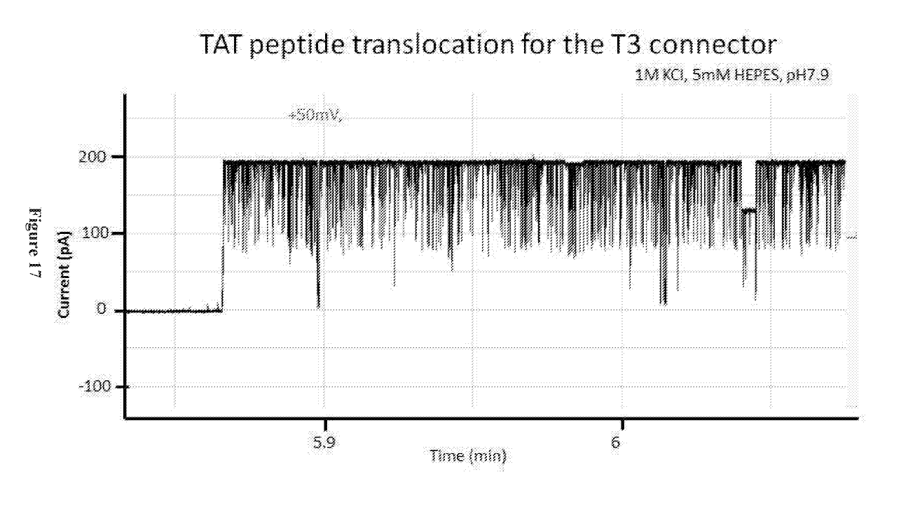

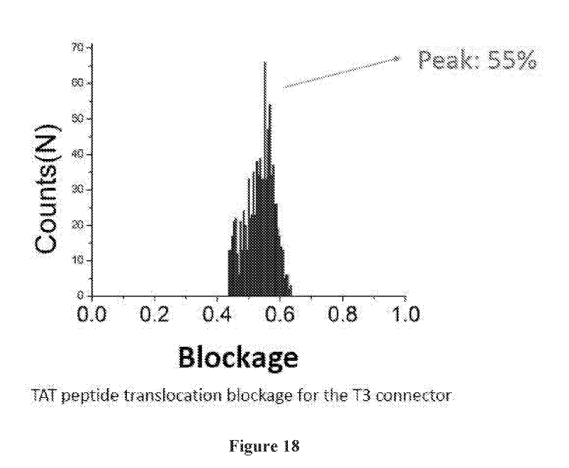

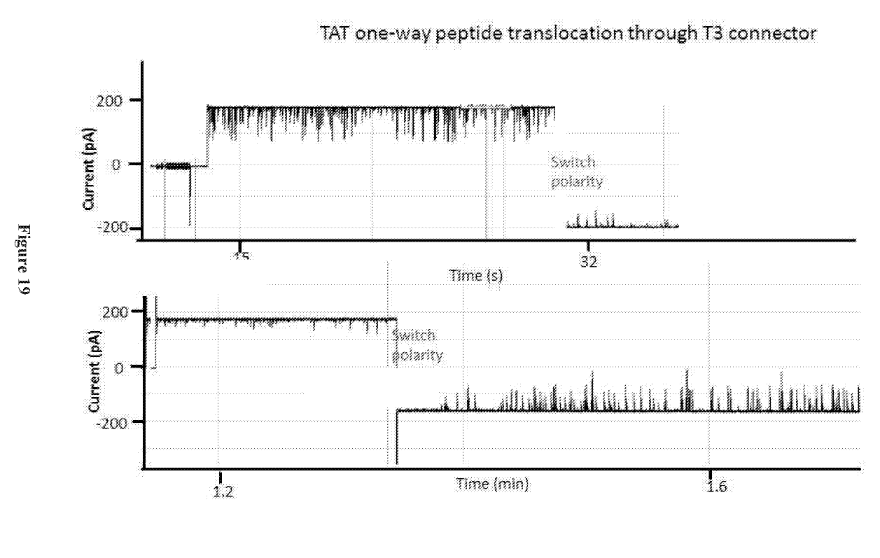

The disclosure below includes Section 1 (which includes the Introduction set forth above, description of FIGS. 1-8, FIGS. 13-27, and description of sequence listing) and Section 2 (which includes description of FIGS. 9-12 set forth above).

Section 1

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes, in some embodiments, a nucleic acid molecule encoding a T3 or T4 connector polypeptide variant. The T3 or T4 connector polypeptide variant comprises an amino acid sequence having at least 90, 91, 92, 93, 94, 95, 96, 97, or 99% sequence identity to a sequence shown in SEQ ID NO:2, or SEQ ID NO:4, respectively. In some embodiments, the nucleic acid molecule encodes a T3 or T4 connector polypeptide variant comprises an amino acid sequence as set forth in SEQ ID NO:2 or SEQ ID NO:4, respectively. In some embodiments, the nucleic acid molecule comprises the sequence shown in SEQ ID NO:1, or SEQ ID NO:3.

A nucleic acid or polypeptide sequence can be compared to another sequence and described in terms of its percent sequence identity. In calculating percent sequence identity, two sequences are aligned and the number of identical matches of nucleotides or amino acid residues between the two sequences is determined The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It will be appreciated that a single sequence can align differently with other sequences and hence, can have different percent sequence identity values over each aligned region. It is noted that the percent identity value is usually rounded to the nearest integer.

The alignment of two or more sequences to determine percent sequence identity is performed using the algorithm described by Altschul et al. (1997, Nucleic Acids Res., 25:3389-3402) as incorporated into BLAST (basic local alignment search tool) programs, available at ncbi.nlm.nih.gov on the World Wide Web. BLAST searches can be performed to determine percent sequence identity between a first nucleic acid and any other sequence or portion thereof aligned using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a sequence disclosed herein (e.g., SEQ ID NOs:1-4) and another sequence, the default parameters of the respective programs are used.

TABLE 1
Conservative Amino Acid Substitutions

| | Representative Conservative Amino |
|---|---|
| Amino Acid | Acids |
| Ala | Ser, Gly, Cys |
| Arg | Lys, Gln, His |
| Asn | Gln, His, Glu, Asp |
| Asp | Glu, Asn, Gln |
| Cys | Ser, Met, Thr |
| Gln | Asn, Lys, Glu, Asp, Arg |
| Glu | Asp, Asn, Gln |
| Gly | Pro, Ala, Ser |

TABLE 1

| | |
|---|---|
| His | Asn, Gln, Lys |
| Ile | Leu, Val, Met, Ala |
| Leu | Ile, Val, Met, Ala |
| Lys | Arg, Gln, His |
| Met | Leu, Ile, Val, Ala, Phe |
| Phe | Met, Leu, Tyr, Trp, His |
| Ser | Thr, Cys, Ala |
| Thr | Ser, Val, Ala |
| Trp | Tyr, Phe |
| Tyr | Trp, Phe, His |
| Val | Ile, Leu, Met, Ala, Thr |

Modifications, including substitutions, insertions or deletions are made by known methods. By way of example, modifications are made by site-specific mutagenesis of nucleotides in the DNA encoding the protein, thereby producing DNA encoding the modification, and thereafter expressing the DNA in recombinant cell culture. Techniques for making substitution mutations at predetermined sites in DNA having a known sequence are well known.

The presently-disclosed subject matter further includes, in some embodiments, a conductive channel-containing membrane, comprising (a) a membrane layer; and (b) a T3 and/or T4 connector polypeptide variant that is incorporated into the membrane layer to form an aperture through which conductance can occur when an electrical potential is applied across the membrane, wherein the T3 and/or T4 connector polypeptide variant is selected from among those disclosed herein.

Embodiments described herein find use in a variety of molecular analytical contexts, including, for example, sensitive detection and characterization of chemical and biochemical analytes for biomedical, clinical, industrial, chemical, pharmaceutical, environmental, forensic, national security, toxicological and other purposes, including any situation where rapid, specific and exquisitely sensitive detection and/or characterization of an analyte (e.g., preferably a soluble analyte that is provided in solution) may be desired. Expressly contemplated are embodiments in which the presently disclosed compositions and methods are used for DNA sequencing, including dsDNA sequencing, high-throughput DNA sequencing, genomics, SNP detection, molecular diagnostics and other DNA sequencing applications, and polypeptide detection and identification. Additional utilities include those described in International Patent Application Publication No. WO2010/062697, which is incorporated herein in its entirety by this reference.

Exemplary analytes thus include nucleic acids such as DNA and RNA (including dsDNA and dsRNA), including for the detection and identification of single nucleotide polymorphisms (SNPs) and/or mutations in such nucleic acids, and/or nucleic acid sequence determination. Other exemplary analytes that may be detected and/or characterized using the herein described compositions and methods include polypeptides and other biopolymers (e.g., proteins, glycoproteins, peptides, glycopeptides, oligosaccharides, polysaccharides, lipids, glycolipids, phospholipids, etc.) and other biomolecules (e.g., soluble mediators, cofactors, vitamins, bioactive lipids, metabolites, and the like), drugs and other pharmaceutical and pharmacological agents, including natural and synthetic compounds, food and cosmetics agents such as flavorants, odorants, preservatives, antioxidants, antimicrobial agents, stabilizers, carriers, excipients, modifying agents and the like, natural and synthetic toxins, dyes, and other compounds.

Accordingly and in certain embodiments, any analyte for which detection and/or characterization is desired may be used, where it will be recognized from the disclosure herein that the analyte is preferably soluble in a solvent that does not compromise the integrity of the particular membrane layer in which the T3 and/or T4 connector polypeptide variant is incorporated to form an aperture through which conductance can occur when an electrical potential is applied across the membrane. Analyte selection may thus vary as a function of the composition of the particular membrane layer being used, which may therefore influence solvent selection. Those skilled in the art will be familiar with criteria to be employed for selecting a solvent that is compatible with a membrane layer of any particular composition. In preferred embodiments, the membrane layer comprises a phospholipid bilayer and the solvent in which the analyte is provided comprises an aqueous solvent, e.g., a solvent that comprises water.

In some embodiments of the conductive channel-containing membrane, the membrane layer comprises a lipid layer. In a further embodiment the lipid layer comprises amphipathic lipids, which in certain still further embodiments comprise phospholipids and the lipid layer comprises a lipid bilayer. In certain other embodiments the lipid layer is selected from a planar membrane layer and a liposome. In certain embodiments the liposome is selected from a multilamellar liposome and a unilamellar liposome. In certain other embodiments the incorporated T3 and/or T4 connector polypeptide variant is mobile in the membrane layer. In certain other embodiments the conductive channel-containing membrane is capable of translocating double-stranded DNA through the aperture when the electrical potential is applied. In certain other embodiments the conductive channel-containing membrane is capable of translocating polypeptides through the aperture when the electrical potential is applied. In certain embodiments conductance occurs without voltage gating when the electrical potential is applied. In some embodiments of the engineered nucleic acid molecule encoding a T3 or T4 viral connector polypeptide variant as disclosed herein can be assembled to a T3 or T4 DNA-packaging motor connector protein, and the T3 and T4 motor connector is incorporated into the lipid membrane layer. In more severe mutations in a T3 or T4 viral connector polypeptide, the structure of T3 or T4 motor connector cannot be assembled into the correct form in lipid membrane layer.

In some embodiments, the T3 and/or T4 connector polypeptide variant comprises a detectable label. In some embodiments, the detectable label is selected from the group consisting of a colorimetric indicator, a GCMS tag compound, a fluorescent indicator, a luminescent indicator, a phosphorescent indicator, a radiometric indicator, a dye, an enzyme, a substrate of an enzyme, an energy transfer molecule, a quantum dot, a metal particle and an affinity label.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

Although any methods, devices, and materials similar or equivalent to those described herein can be used in the practice or testing of the presently-disclosed subject matter, representative methods, devices, and materials are now described.

The presently-disclosed subject matter is further illustrated by the following specific but non-limiting examples. The following examples may include compilations of data that are representative of data gathered at various times during the course of development and experimentation related to the present invention.

The presently disclosed subject matter relates to an engineered DNA-packaging motor protein connector and method of use thereof. More particularly, the presently disclosed subject matter relates to an engineered DNA-packaging motor protein connector that can be incorporated into a lipid membrane to form an electroconductive aperture, for use in DNA translocation and other applications.

The presently disclosed subject matter includes an isolated conductive channel-containing membrane selected from T3 and T4 connector channel protein, including one or more amino acid mutations and/or deletions and/or insertions relative to wild type.

The presently disclosed subject matter further includes a membrane includes a lipid bilayer membrane, and an isolated DNA-packaging motor connector protein that is incorporated into the membrane layer to form an aperture through which conductance can occur when an electrical potential is applied across the membrane. In some embodiments, in the conductive channel-containing membrane, the DNA-packaging motor connector protein is a T3 and/or T4 connector channel protein. In some embodiments, the T3 and/or T4 connector channel protein including one or more amino acid mutations and/or deletions and/or insertions relative to wild type.

In some embodiments, the T3 and/or T4 connector channel protein comprises a homododecamer of viral DNA-packaging motor connecting protein polypeptide subunits. In some embodiments, the subunit of the homododecaamer comprises an affinity/alignment domain. In some embodiments, the affinity/alignment domain comprises a polypeptide of (i) a Strep-11 tag, (ii) a polyhistidine polypeptide tag of 3, 4, 5, 6, 7, 8, 9, 10, 11 or 12 contiguous histidine residues, (iii) a polyarginine polypeptide of 3, 4, 5, 6, 7, 8, 9, 10, 11 or 12 contiguous arginine residues, (iv) an HIV Tat polypeptide of sequence YGRKKRRQRR, or (v) a peptide tag of sequence DRATPY.

In some embodiments, each subunit of the homododecamer of viral DNA-packaging motor connector protein polypeptide comprises a polypeptide of all or a transmembrane aperture-forming portion of T4 DNA-packaging motor connector protein polypeptide. In a specific embodiments, each subunit comprises a polypeptide of SEQ ID NO: 4. In a specific embodiments, each subunit comprises a polypeptide of SEQ ID NO: 3. In some embodiments, the membrane is capable of translocating double-stranded DNA through the aperture when the electrical potential is applied.

Further provided, in some embodiments of the presently disclosed subject matter, is a method of making a conductive channel-containing membrane. The method includes the steps of (a) preparing dried amphipathic lipids on a solid substrate by contacting a first solution comprising amphipathic lipids and an organic solvent with the solid substrate and substantially removing the solvent; and (b) resuspending the dried amphipathic lipids in a second solution that comprises an aqueous solvent, an osmotic agent and a plurality of isolated viral DNA-packaging motor connector protein subunit polypeptides that are capable of self-assembly into a homododecameric viral DNA-packaging motor connector protein, to obtain a membrane that comprises a lipid bilayer in which is incorporated the viral DNA-packaging motor connector protein under conditions and for a time sufficient for said connector protein to form an aperture through which conductance can occur when an electrical potential is applied across the membrane, and thereby making a conductive channel-containing membrane.

Still further, in some embodiments, the presently disclosed subject matter provides a method of concentrating nucleic acid molecules on one side of a conductive channel-containing membrane.

The method includes the steps of (a) making a conductive channel-containing membrane by a method comprising: (i) substantially removing solvent from a mixture comprising amphipathic lipids and at least one solvent, to obtain dried amphipathic lipids; and (ii) resuspending the dried amphipathic lipids in a second solution that comprises an aqueous solvent, an osmotic agent and a plurality of isolated viral DNA-packaging motor connector protein subunit polypeptides that are capable of self-assembly into a homododecameric viral DNA-packaging motor connector protein, to obtain a membrane that comprises a lipid bilayer in which is incorporated the viral DNA-packaging motor connector protein under conditions and for a time sufficient for said connector protein to form an aperture through which conductance can occur when an electrical potential is applied across the membrane, and thereby making a conductive channel-containing membrane; and (b) contacting the conductive channel-containing membrane of (a) with one or a plurality of nucleic acid molecules and with an electrical potential that is applied across the membrane, under conditions and for a time sufficient for electrophoretic translocation of the nucleic acid through the aperture of the connector protein, and thereby concentrating nucleic acid molecules on one side of the conductive channel-containing membrane.

The presently-disclosed subject matter is further illustrated by the following specific but non-limiting examples. Some of the following examples are prophetic, notwithstanding the numerical values, results and/or data referred to and contained in the examples.

### EXAMPLES

DNA translocases, a class of biological motors in cells, bacteria, and viruses, are essential for cellular processes, such as DNA replication, DNA repair, homologous recombination, cell mitosis, bacterial binary fission, Holliday junction resolution, viral genome packaging, RNA transcription, and nuclear pore transport. The channel of DNA translocase of double-stranded DNA viruses allows viral genomic DNA to enter the protein procapsid shell during viral maturation and to exit during host infection. It was recently showed that the DNA translocase of bacteriophage phi29 uses the mechanism of “Revolution through a one-way valve” and T4 uses “Torsional compression”. This raises a question of how dsDNA is ejected during infection if the channel acts like one-way inward valve. Here we report the finding of three steps of conformational changes of the portal channel that is common to DNA translocases of bacterial virus T3 and T4. All channels of these motors exercise three discrete steps of gating, with each step resulting in 33% reduction of channel dimension per step, as revealed by single channel electrophysiological assay. These findings led to the conclusion that the three steps of gating is due to three steps of channel conformational changes, which concurs with the previous publications of three major bands arising from quantized DNA packaging intermediates. This supports the speculation that the one-way inbound channel during the DNA packaging process was transformed into an outbound channel during DNA ejection process. This finding will lead to the slight twisting of the dsDNA by a motor using revolution mechanism without rotation.

Materials and Methods

Materials and Reagents

The phospholipid, 1,2-diphytanoyl-sn glycerol-3-phosphocholine (DPhPC) (Avanti Polar Lipids), n-Decane (Fisher), chloroform (TEDIA) were used as instructed by the vendor. All other reagents were from Sigma, if not specified. Lipid A: 5% (wt/vol) DPhPC in Hexane. Lipid B: 20% (wt/vol) DPhPC in Decane. His binding buffer: 15% glycerol, 0.5 M NaCl, 5 mM Imidazole, 10 mM ATP, 50 mM Tris-Cl, pH 8.0). His washing buffer: 15% glycerol, 500 mM NaCl, 50 mM Imidazole, 10 mM ATP, 50 mM Tris-Cl, pH 8.0. His elution buffer: 15% glycerol, 500 mM NaCl, 500 mM Imidazole, 50 mM ATP, 50 mM Tris-Cl, pH 8.0.

Expression and Purification of T3 and T4 Connectors

Gene gp8 encoding T3 connector protein was synthesized (Genescript) and then cloned into pET3c vector between NdeI and BamHI separately. A 6×His-tag was inserted into the C-terminal for purification. Plasmid pET3c harboring gp8 was transformed into E. Coli BL21(DE3) separately and single colony was cultured in 10 mL Luria-Bertani medium (LB) overnight at 37° C. Then the cultured bacteria were transferred to 1 L of fresh LB medium. 0.5 mM IPTG was added to the medium to induce protein expression when OD600 reached 0.5-0.6. After 3 hours culture, cells were collected by centrifugation at 6000×rpm for 15 min and the pellet was resuspended with His Binding Buffer. Bacteria were lysed by passing through French press and the clear supernatant was collected after 12000×rpm for 20 min centrifugation and loaded to His•Bind® Resin Column. T3 connector protein was eluted with His Elution buffer after several rounds washing from the His•Bind® Resin Column.

T4 gp20 gene encoding connector protein was amplified from T4 genome and cloned into pET3c Vector at NdeI site and BamHI site (Keyclone). A 6×His-tag was also introduced at the C-terminus for purification. Due to its hydrophobic property, T4 connector easily aggregates. The expression and purification procedure is modified from a previous publication (38). Plasmid pET3c harboring gp20 was transformed into E. coli HMS174(DE3) and single colony was cultured in 10 ml LB medium overnight at 37° C. Then the cultured bacteria were transferred to 1 L of fresh LB medium and cultured until OD reached 0.5-0.6. Then, 0.5 mM IPTG final concentration was added to induce T4 connector expression and the culture was changed to 18° C. and continued overnight. Cells were harvested by centrifugation at 6000×rpm for 15 min and resuspended in His binding buffer. The cells were lysed by passing through French press. Cell pellet was collected after centrifugation at 12000×rpm for 20 min and resuspended with His binding buffer containing 1% N-Lauroylsarcosine for 20 min. The supernatant was collected after centrifugation at 12000×rpm 1 hr and loaded to His•Bind® Resin Column and eluted after several rounds washing. All the final protein products were verified by 10% SDS-PAGE gel.

Preparation of Lipid Vesicles Containing the Connector.

All connector/liposome complexes were prepared following the procedure published previously (29, 37). Briefly, 0.5 ml of 1 mg/ml DPhPC in chloroform was added to a round bottomed flask and the chloroform was evaporated under vacuum using the Rotary Evaporator (Buchi). The dried lipid film was rehydrated with 0.5 ml of connector protein solution containing 250 mM sucrose. Unilamellar lipid vesicles were obtained by extruding the lipid solution through a 400 nm polycarbonate membrane filter (Avanti Polar Lipids).

Connector Insertion into Planar Lipid Bilayer

The insertion into a lipid bilayer with connector reconstituted liposomes has been reported previously (29, 37). Briefly, a thin Teflon partition with an aperture of 200 μm was used to separate the Bilayer Lipid Membrane (BLM) cell into cis- and trans-compartments. The aperture was pre-painted with 5% (wt/vol) DPhPC in hexane solution. The cis and trans-chambers were filled with conducting buffer, 1 M KCl, 5 mM HEPES, pH 7.8. Then 20% (wt/vol) DPhPC in n-decane solution was used to form lipid bilayer. After confirming the formation of the lipid bilayer, the connector/liposome complexes were added to the cis-chamber to fuse with the planar lipid bilayer to form the membrane embedded nanopore.

Electrophysiological Measurements

A pair of Ag/AgCl electrodes inserted to both compartments was used to measure the current traces across the BLM. The current trace was recorded using an Axopatch 200B patch clamp amplifier coupled with the BLM workstation (Warner Instruments) or the Axon DigiData 1440A analog-digital converter (Axon Instruments). All voltages reported were those of the trans-compartment. Data was low band-pass filtered at a frequency of 5 kHz or 1 KHz and acquired at a sampling frequency of 2 KHz. The PClamp 9.1 software (Axon Instruments) was used to collect the data, and the software Origin Pro 8.0 was used for data analysis.

Results

Cloning, Expression and Purification of the Connectors.

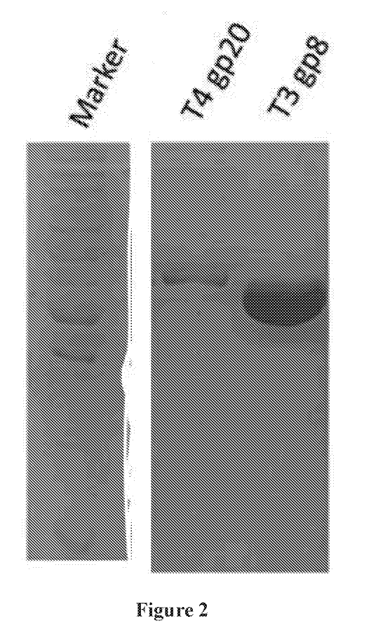

A 6×His tag was inserted into the C-terminus of the T4 and T3 connector channels to facilitate the purification. A 6× amino acid glycine linker was introduced between connector and His tag to provide end flexibility. T4 connector showed a strong tendency to aggregate due to its hydrophobic nature. Therefore, 1% N-Lauroylsarcosine surfactant was added to the purification buffer to solubilize the protein (38). After purification to homogeneity, the protein was run in 10% SDS-PAGE. The single protein subunit of T4 and T3 connector corresponded to the predicted molecular weights of 60 kDa and 59 kDa, respectively (FIG. 2).

EM study revealed that T4 connector exists as a dodecameric ring, with a contour dimension of 14 nm long and 7 nm wide, and about ˜3 nm in diameter in the interior of the channel (43); however, currently no crystal structure is available. Several studies revealed that T3 connector protein exists as a mixed population of 12 and 13 subunits. The percentages of these two oligomer states vary in each culture growth indicating that assembly of the connector protein depends on the expression conditions and other factors (44-46). EM studies revealed that the three dimensional structure of T3 connector is: 14.9 nm at external diameter; 8.5 nm in height; average 3.7 nm of internal open channel (44).

Insertion of Connector Channels into Lipid Membrane for Determining Channel Size Using Conductance Measurements

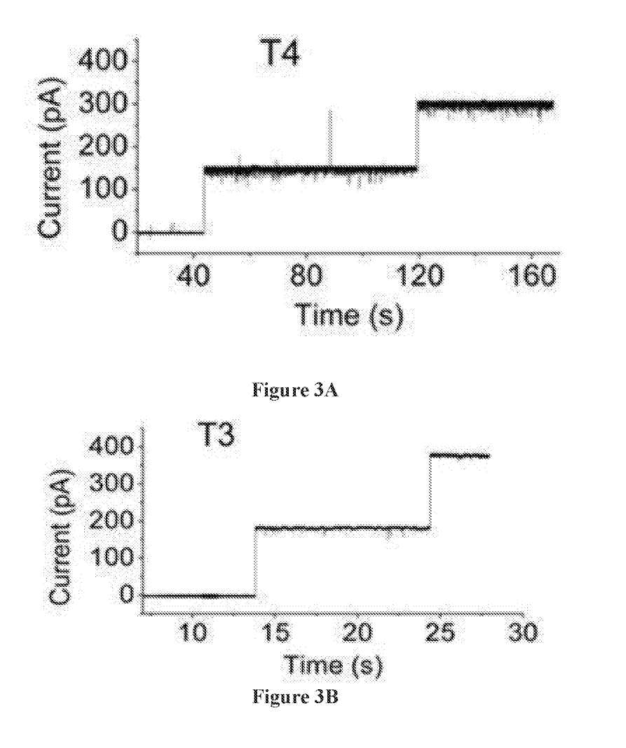

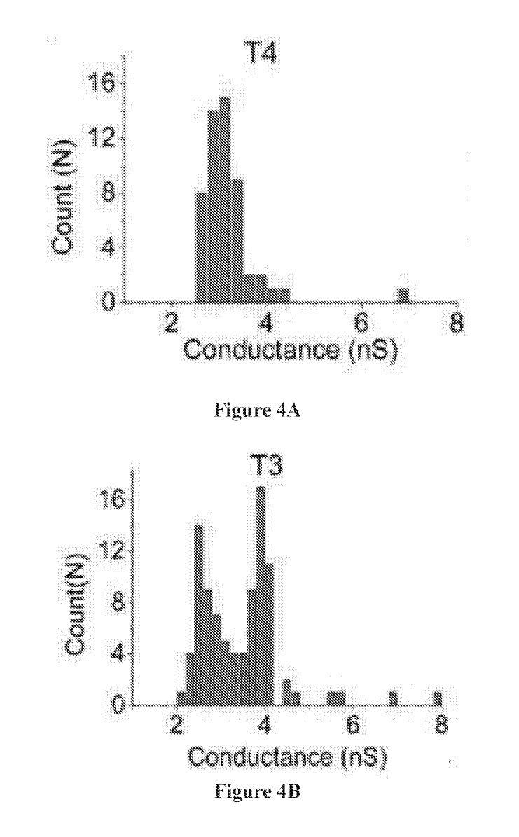

To incorporate T4 and T3 connector into planar lipid bilayer, we adopted a two-step procedure: reconstitution of connector in liposomes, followed by fusion of proteoliposomes into planar lipid bilayer to form the membrane-embedded connector, as described previously (29). The current jump for each channel insertion was measured at a fixed voltage to determine the channel sizes of T3 and T4 connector channels (FIG. 3A, FIG. 3B). The experiments were carried out using the same buffer, 1 M KCl, 5 mM HEPES, pH 7.8, under 50 mV applied potential. The channel conductance (derived from the ratio of measured current jump with the applied voltage) of T4 was determined to be 4.52±0.33 nS, 4.10±0.22 nS, and 3.03±0.37 nS (FIG. 4A). T3 conductance distribution appeared as two peaks: 2.65±0.31 nS and 3.90±0.38 nS (FIG. 4B).

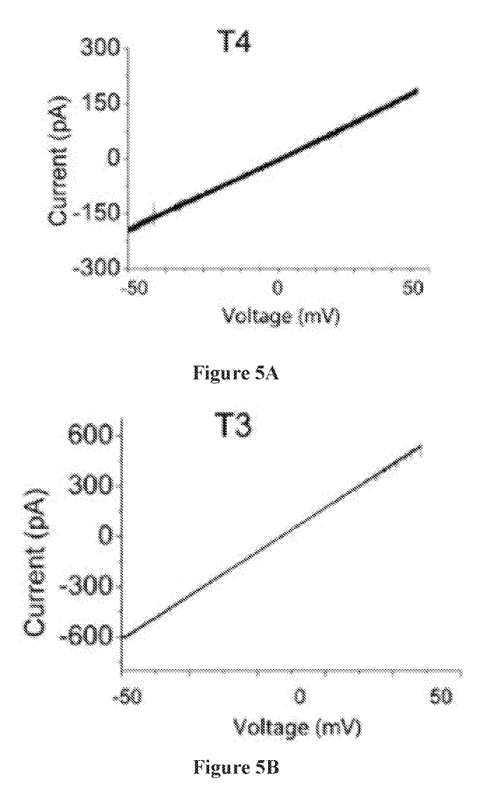

Under a scanning voltage (−50 mV to 50 mV; 2.2 my/s), T4 and T3 connector channels all display a linear Current-Voltage (I-V) relationship without voltage gating phenomenon (FIG. 5A, FIG. 5B). When 100 mV was applied, T4 and T3 connector channels remained stable without displaying voltage gating.

Three Step Gating of Connector Channels

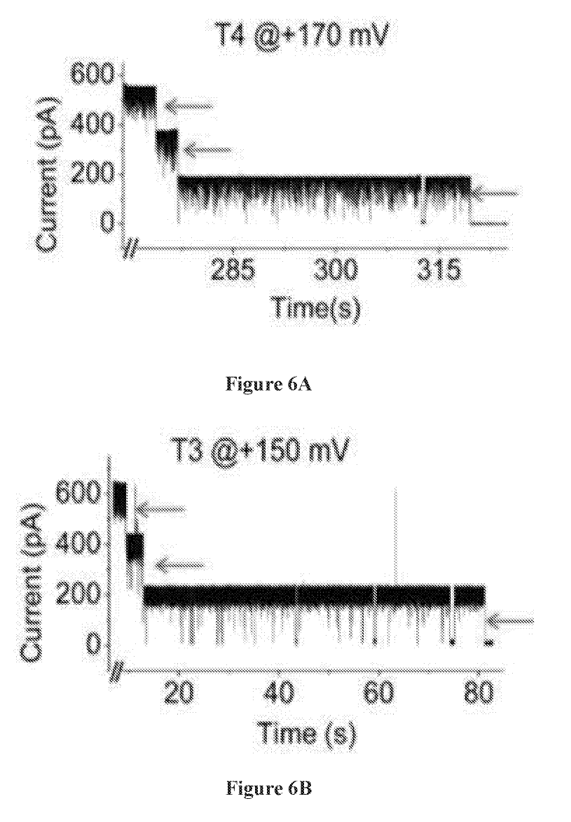

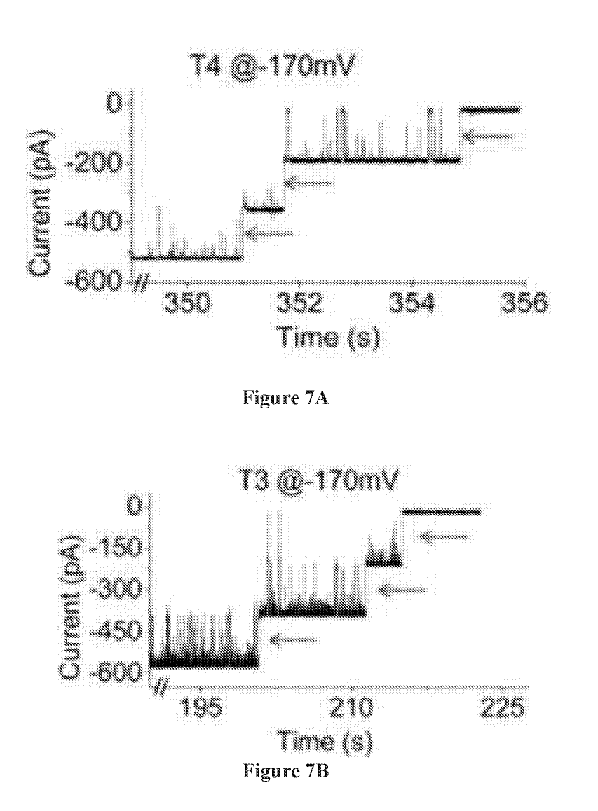

When a higher voltage (>100 mV) was applied, three distinct steps of conformational change of the channel were observed in all four connector channels. The conformational change of the channel was reflected in the reduction of electrical current of 33%, 66% and 99% for the first, second, and third step, respectively (FIG. 6A, FIG. 6B). Three discrete steps gating of T4 and T3 connector channel was found under applied positive voltage 170 mV and 150 mV (FIG. 6A, FIG. 6B). Similar phenomenon was observed under negative voltages of −175 mV and −125 mV (FIG. 7A, FIG. 7B). These are the minimum voltage required for the channels of gating.

Comparisons of Steps Between Channel Gating and Quantized DNA Packaging or Ejection

Previous investigation of viral DNA packaging or ejection using different methods have revealed quantized packaging of DNA by analyzing the length of incompletely packaged DNA in different bacteriophages (16, 17). Such quantized DNA packaging phenomenon has been a puzzle for a long time. Here we tried to link the previously unexplained quantized packaging data with our new finding of the conformational change.

Discussion

All connector channels of dsDNA bacteriophages display left-handed channel wall to facilitate one-way traffic during dsDNA translocation into pre-assembled protein shells by a revolution mechanism without rotation (11, 47-49). This raises a question of how dsDNA is ejected to enter the infected cell if the channel is a one-way inward valve. The conformational changes of the channel have been reported previously (20, 35). For example, it has been shown that the conformation change of phi29 connector was induced by DNA, pRNA or divalent metal ions as revealed by circular dichroism and quenching of the intrinsic tryptophan fluorescence (18, 19). In our current study, three steps of conformational changes for the connector of T4 and T3 were observed. Such conformational changes would allow conversion of the left-handed connector after completion of DNA packaging towards the opposite configuration, thus facilitating DNA one-way ejection into host cells for infection. In the Phi29 crystal structure, the connector subunit displays a left-handed 30° tilt (49, 50). Cryo-EM has also revealed the conformational difference between the free in vitro connector and the connector in the DNA-filled virion (20). It was reported that when treated as a rigid body, the connector crystal structure does not fit into the connector in the Cryo-EM density maps, as shown by a correlation coefficient as low as 0.55. The correlation coefficient was improved to 0.70 after manual adjusting, resulting in a 10° twist of the connector towards the connector axis (20). Conformational changes of connectors have also been reported in other bacteriophages systems (41, 51-55). It was found that the N-terminal external region underwent significant conformational shift in the DNA-filled capsid (20). It was concluded that angular twisting and restructuring of the connector core subunit are promoted by the interactions among Phi29 DNA and its structural proteins (20). Due to the association and alignment of the dsDNA with wall of the connector channel (47, 48, 50, 56, 57) and the relatively stationary nature of the internal wider C-terminal region, a noteworthy conformational shift in the external narrow N-terminal region could result in a clockwise twist of the dsDNA when viewed from the C- to N-terminus. As evidenced above (20), if the N-terminal external region is shifted more significantly than the internal C-terminal region, a leftward twist of the DNA will occur during revolution along the connector channel. This is in agreement with the observed clockwise twist of 1.5 degree per nucleotide relative to the C-terminus of the connector (13). In addition, the reported increase in the frequency of DNA twisting per nucleotide with increase in capsid filling, is in agreement with the observation that the conformational change of the channel accelerates towards the end of the packaging process (35). This is logical since the dsDNA is aligned with the wall of the connector channel, and when DNA packaging, or ejection is close to completion, a final conformation will be adopted.

The conductance is normally a reflection of the narrowest constriction of the channel (29), but other factors such as the length of the cylinder region can contribute (58). In one of the early studies on MspA, the conductance of the wild-type is about 4.9 nS, whereas the conductance reduced by a factor of 2-3 after the change of three amino acids D into N (59). Another possible variation is due to the oligomeric state of T4 connector resulting in a heterogeneous populations with wider conductance distribution. Although it is believed that the T4 connector exists as dodecamers exclusively in the biologically active state (60), the stoichiometry of the connector of different bacteriophages has been reported to vary from 11-mer to a 14-mer in vitro following ectopic expression and assembly (46, 61-64). Several studies revealed that T3 connector protein exists as a mixed population of 12 and 13 subunits. The percentages of these two oligomer states vary in each culture growth indicating that assembly of the connector protein depends on the expression conditions and other factors (44-46). It has been reported that the conformational changes occurring in specific segments, such as helix α6 of the tunnel loop and the crown region may be responsible for the different oligomeric states (41).

Conclusions

The motor channel of T3 and T4 display three discrete step of voltage gating resulting from channel conformational changes. The three steps of gating coincide with the three major steps of quantized DNA packaging as reported previously; suggesting that the one way inbound channel during the DNA packaging process is transformed into an outbound channel prepared for DNA ejection during the host infection.

Finally, for further explanation of the features, benefits and advantages of the present invention, attached hereto is Appendix A, which is incorporated herein by this reference.

Throughout this document, various references are mentioned. All such references are incorporated herein by reference, including the references set forth in the following list:

Section 2

Abstract

The DNA packaging motor of dsDNA bacterial viruses contains a head-tail connector with a channel for genome to enter during assembly and to exit during host infection. The DNA packaging motor of bacterial virus phi29 was recently reported to use the “One-way Revolution” mechanism for DNA packaging. This raises a question of how dsDNA is ejected during infection if the channel acts as a one-way inward valve. Here we report a three step conformational change of the portal channel that is common among DNA translocation motors of bacterial viruses T3, T4, SPP1, and phi29. The channels of these motors exercise three discrete steps of gating, as revealed by electrophysiological assays. It is proposed that the three step channel conformational changes occur during DNA entry process, resulting in a structural transition in preparation of DNA movement in the reverse direction during ejection.

Introduction

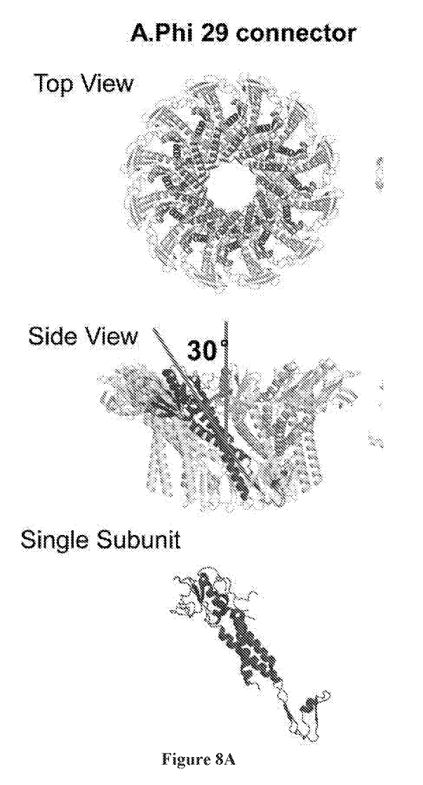

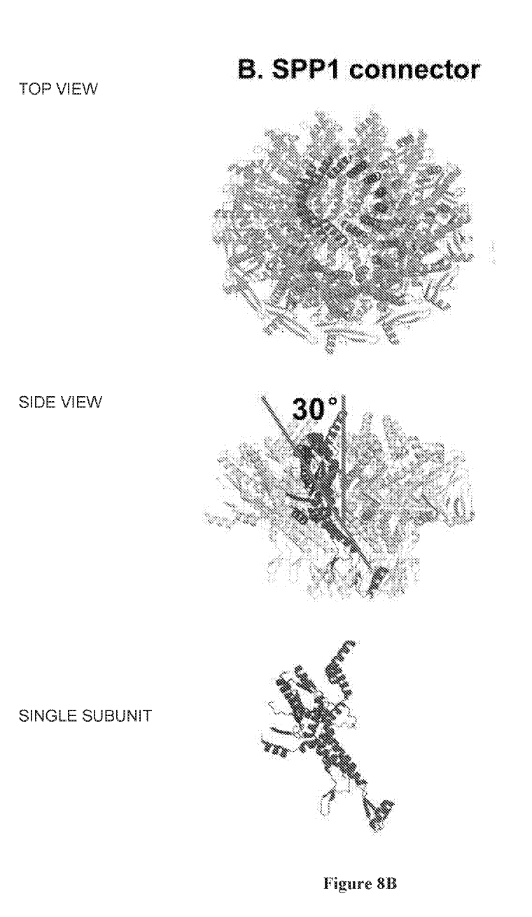

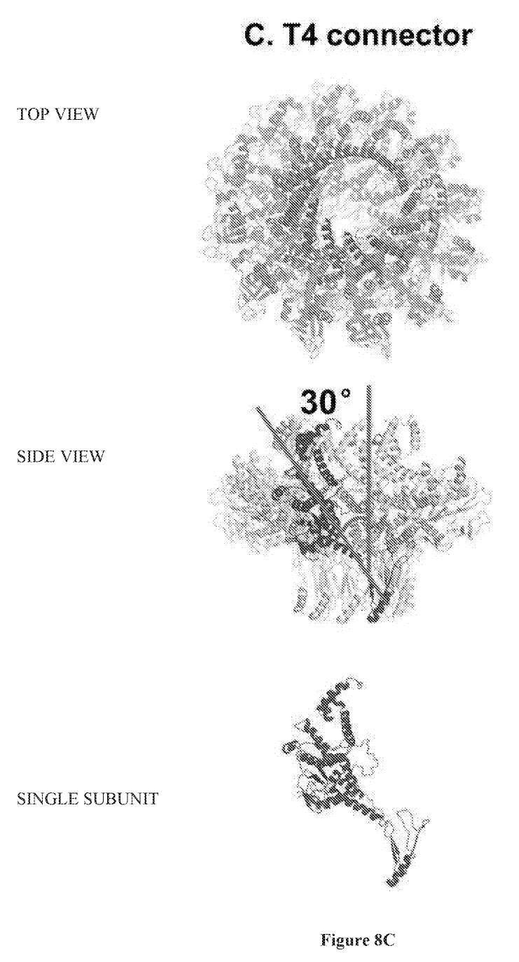

DNA translocation motors are ubiquitous in living systems (Guo et al., 2016). During replication, the genome of double stranded DNA (dsDNA) viruses is packaged into a preformed protein shell, referred to as the procapsid. This process requires a powerful, ATP-driven packaging motor. In many viruses, the motor involves a pair of DNA packaging proteins, a smaller auxiliary subunit is usually a protein oligomer that comes into contact with the dsDNA, and a larger one is an ATPase protein (Guo et al., 1987). In many dsDNA bacterial viruses and herpes viruses, the motor docks onto a structure called the portal or connector. Structural studies have shown that the portals in herpes virus and a variety of tailed bacterial viruses, such as phi29, SPP1, T4, and T3, share a similar cone-shaped dodecameric structure (FIG. 8), even though their primary sequences do not display any significant homology.

In bacterial virus phi29, the portal is comprised of 12 protein subunits assembled into a truncated cone structure, with a diameter of 13.8 nm and 6.6 nm at the wide and narrow ends, respectively. The interior channel is 3.6 nm at the narrowest constriction (41). In SPP1, the assembled channel is a 13-mer structure, and the narrowest part is 2.77 nm in diameter (FIG. 8) (42, 43). The T4 portal exists as a dodecameric ring that is 14 nm long and 7 nm wide, and an interior channel of −3 nm in diameter (Sun et., al, Nat. Commum. 6 7548). The T3 portal is a mixture of 12 and 13 subunits, depending on the protein expression conditions and other factors. The 12-mer version of the T3 portal is 14.9 nm in width, 8.5 nm in height and 3.7 nm in diameter for the internal channel (46).

The portal plays a critical role in genome packaging and the ejection process. During assembly, it acts as a docking point for the motor ATPase and a conduit for dsDNA entry. After DNA packaging, the portal serves as a binding site for the tail components in order to complete virion assembly. When bacterial viruses initiate infection, DNA is ejected through the coaxial channel of the portal and tail channel into the host cell. In the bacterial virus SPP1, the portal protein undergoes a concerted structural conformational change during its interaction with DNA (Chaban et al., 2015). Recent results obtained using the membrane-embedded phi29 portal connector demonstrated that dsDNA moves in only one direction, i.e. from the external narrow end to the internal wide end, referred to as “one-way traffic” (Jing et al., 2010). Biological data from the ATPase studies combined with single molecule studies led to the conclusion that the DNA translocation of bacterial virus phi29 takes place via a “Push through a one-way valve” (Zhang et al., 2012) or a “One-way revolution mechanism” in order to package DNA (Schwartz et al., 2013; Guo, 2014; De-Donatis et al., 2014). The meaning of “Push” is in accordance with the findings in T4 that indicate a compression mechanism (Ray et al., 2010; Dixit et al., 2012; Harvey, 2015). Since the channels act like a one-way valve, an obvious question arises: how is dsDNA ejected during the course of infection if the channel is a one-way inward valve? Earlier studies demonstrated that the portal exercises conformational changes during the respective DNA packaging and ejection processes. For example, in the phi29 portal, conformational changes are inducible by DNA, pRNA or divalent metal ions (Geng et al., 2013; Urbaneja et al., 1994; Tolley et al., 2008). It was also reported that, the channel loop of phi29 DNA packaging motor plays an important function near the end of packaging to retain the DNA (Grimes et al., 2011). Cryo-EM imaging also revealed conformational changes of the connector in infectious virion in comparison with the free connector in vitro (Tang et al., 2008). However, none of these studies have yet shown conformational changes at the single molecule level.

Nanopore-based single molecule detection has attracted considerable attention across a number of disciplines due to its versatility of application. Examples include the detection of small molecules of chemicals, nucleotides, drugs and enantiomers, as well as larger polymers, such as PEG, polypeptides, RNA and DNA. One novel application was the insertion of the phi29 portal into an artificial membrane in order to serve as a robust nanopore (Wendell et al., 2009) for single molecule detection (Hague et al., 2012) and disease diagnosis (Wang et al., 2013). The phi29 portal channel displays voltage-induced channel gating as well as a one-way traffic for dsDNA translocation during the course of DNA packaging (Geng et al., 2011; Jing et al., 2010; Fang et al., 2012). It has been reported that interaction of ligand with the C-terminal of the connector leads to the conformational changes in the phi29 connector channel, resulting in an altered current signal that have been utilized for detecting single antibodies as a very sensitive method for disease diagnosis (Wang et al., 2013). Here we report that the discrete conformational changes in the channel are common in bacterial viruses T3, T4, SPP1 and phi29. These observations support the idea that the one-way inbound channel is transformed into an outbound channel in preparation for DNA ejection (Hu et al., 2013).

Materials and Methods

Materials and Reagents

The phospholipid, 1,2-diphytanoyl-sn glycerol-3-phosphocholine (DPhPC) (Avanti Polar Lipids), n-Decane (Fisher), chloroform (TEDIA) were used as instructed by the vendors. If not specified, other reagents were purchased from Sigma. His binding buffer: 15% glycerol, 0.5 M NaCl, 5 mM Imidazole, 10 mM ATP, 50 mM Tris-Cl, pH 8.0. His washing buffer: 15% glycerol, 500 mM NaCl, 50 mM Imidazole, 10 mM ATP, 50 mM Tris-HCl, pH 8.0. His elution buffer: 15% glycerol, 500 mM NaCl, 500 mM Imidazole, 50 mM ATP, 50 mM Tris-Cl, pH 8.0.

Expression and Purification of Phi29, SPP1, T3, and T4 Portals

The expression and purification of phi29 portal followed the procedure reported previously (Hague et al., 2013a; Wendell et al., 2009). Gene 6 encoding for SPP1 portal protein gp6, and gene 8 encoding for T3 portal protein gp8 were synthesized (Genescript) and then cloned separately into pET3c vector between the NdeI and BamHI sites. A 6×His-tag was inserted at the C-terminus for purification. The resulting plasmids harboring gene 6 or gene 8 were transformed separately into E. Coli BL21(DE3) and a single colony was cultured in 10 mL Luria-Bertani (LB) medium overnight at 37° C. The culture was transferred to 1 L of fresh LB medium and 0.5 mM IPTG was added to induce protein expression after the OD₆₀₀ reached 0.5-0.6. After 3 hrs, cells were collected by centrifugation at 6000×rpm for 15 min and the pellet was resuspended in His Binding Buffer. Bacteria were lysed by passing through a French press and the clear supernatant was collected after centrifugation at 12000×rpm for 20 min and then loaded onto a His•Bind® Resin Column. SPP1 or T3 portal protein was eluted from the His•Bind® Resin Column with His Elution buffer after several rounds of washing.

Gene 20 encoding for the T4 portal protein gp20 was amplified from the T4 genome and cloned into pET3c at the NdeI and BamHI sites (Keyclone). A 6×His-tag was introduced at the C-terminus for purification. Due to its hydrophobicity, T4 portal had a tendency to easily aggregate. Protein expression and purification methods were therefore modified (Quinten et al., 2012). Plasmid pET3c harboring gene 20 was transformed into E. Coli HMS174(DE3) and a single colony was cultured in 10 ml LB medium overnight at 37° C. The culture was transferred to 1 L of fresh LB medium and cultured until OD₆₀₀ reached 0.5-0.6. IPTG (0.5 mM final concentration) was then added to induce T4 portal protein expression. The culture was transferred to 18° C. and incubation continued overnight. Cells were harvested by centrifugation at 6000×rpm for 15 min and resuspended in His binding buffer. Cells were lysed by passing through a French press. The cell pellet was collected after centrifugation at 12000×rpm for 20 min and resuspended in His binding buffer containing 1% N-Lauroylsarcosine for 20 min. The supernatant was collected after centrifugation at 12000×rpm for 1 hr and loaded to His•Bind® Resin Column and eluted after several rounds of washing. The purity of all final protein products was verified by 10% SDS-PAGE gel.

Preparation of Liposomes Containing the Phi29, SPP1, T4 and T3 Portals

All portal/liposome complexes were prepared following our reported procedures (Wendell et al., 2009; Haque et al., 2013a). Briefly, 0.5 mL of 1 mg/mL DPhPC in chloroform was added to a round bottom flask and the chloroform was evaporated under vacuum using a Rotary Evaporator (Buchi). The dried lipid film was rehydrated with 0.5 mL of portal protein solution containing 250 mM sucrose. Unilamellar lipid vesicles were obtained by extruding the lipid suspension through a 400 nm polycarbonate membrane filter (Avanti Polar Lipids).

Portal Insertion into Planar Lipid Bilayer

Procedures for inserting the portal connector into a lipid bilayer have been reported previously (Cal et al., 2008; Haque et al., 2013a; Wendell et al., 2009). Briefly, a thin Teflon partition with an aperture of 200 μm was used to separate the Bilayer Lipid Membrane (BLM) chamber into cis- and trans-compartments. The aperture was pre-painted with 5% (wt/vol) DPhPC in hexane solution. The cis and trans-chambers were filled with conducting buffer, 1 M KCl, 5 mM HEPES, pH 7.8. Then 20% (wt/vol) DPhPC in decane solution was used to form a planar lipid bilayer. After confirming the formation of the lipid bilayer, the portal/liposome complexes were added to the cis-chamber to fuse with the planar lipid bilayer to form the membrane embedded nanopore.

Electrophysiological Measurements

The stochastic nanopore sensing technique is based on the principle of the classical Coulter Counter or the ‘resistive-pulse’ technique (Coulter, 1953). The portal is located in an electrochemical chamber, which is separated into two compartments filled with conducting buffers. Under an applied voltage, ions passing through the portal channel will generate current in pico-Ampere (pA) scale (Hague et al., 2013b). When a charged molecule passes through the channel, it will generate transient current blockages due to volumetric exclusion of ions from the pore. Various parameters, such as the event dwell time, current amplitude, and unique electrical signature of the current blockages can be used either individually or in combination for detection.

A pair of Ag/AgCl electrodes inserted into both compartments was used to measure the current traces across the BLM. The current trace was recorded using an Axopatch 200B patch clamp amplifier coupled with the BLM workstation (Warner Instruments) or the Axon DigiData 1440A analog-digital converter (Axon Instruments). All voltages reported were those of the trans-compartment. Data was low band-pass filtered at a frequency of 5 kHz or 1 kHz and acquired at a sampling frequency of 2-20 KHz. PClamp 9.1 software (Axon Instruments) was used to collect the data, and Origin Pro 8.0 was used for data analysis.

Results

Cloning, Expression and Purification of the Portals of Phi29, SPP1, T4 and T3

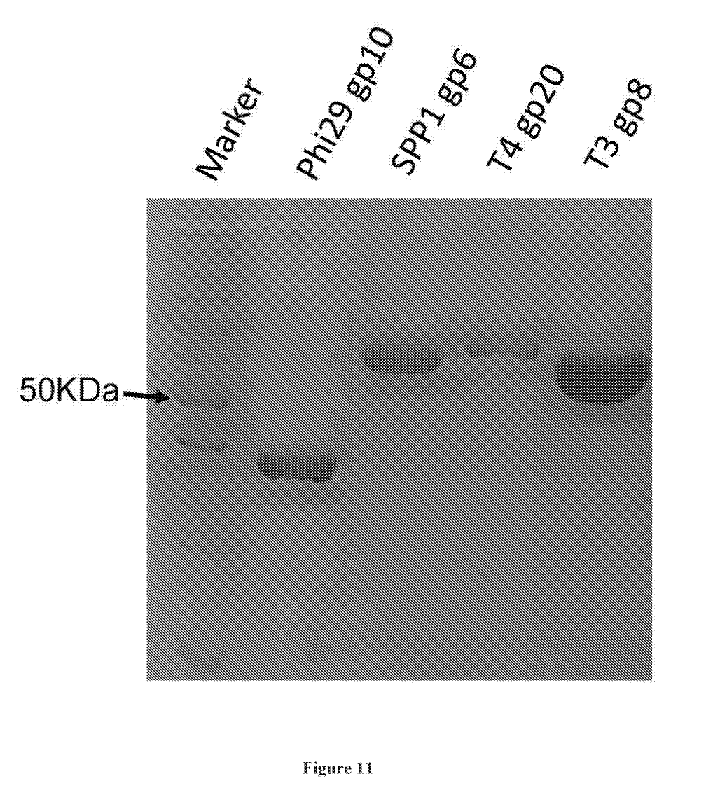

Following the strategy previously used for the purification of phi29 portal (Cal et al., 2008; Haque et al., 2013a; Wendell et al., 2009), a 6× glycine linker was introduced between the portal coding sequence and 6×His-tag to provide end flexibility. Both SPP1 and T3 portals were soluble in the cytoplasm of E. Coli. The T4 portal showed a strong tendency to aggregate due to its hydrophobic nature. Therefore, 1% N-Lauroylsarcosine surfactant was added to the purification buffer to solubilize the protein (Quinten et al., 2012). After purification to homogeneity, proteins were analyzed by 10% SDS-PAGE. The single protein subunit of the phi29, SPP1, T4 and T3 portals corresponded to their predicted sizes of 36 kDa, 56 kDa, 60 kDa and 59 kDa, respectively (FIG. 11).

Insertion of Portal Channels into Lipid Membrane for Determining Channel Size Using Conductance Measurements

To incorporate phi29, SPP1, T4 and T3 portal proteins into a planar lipid bilayer, we adopted a two-step procedure described previously (Wendell et al., 2009): reconstitution of the portal in liposomes, followed by fusion of protein/liposomes with the planar lipid bilayer to form the membrane-embedded portal channel. Experiments were carried out using 1 M KCl, 5 mM HEPES, pH 7.8 conduction buffer and 50 mV applied potential. Each current jump represented the insertion of one channel into the lipid bilayer. Since the fusion of the portal protein/liposome with the planar lipid bilayer is a random event, the time between independent insertion events varies. FIG. 9A-D provides representative results for the portals of the four phages. The channel conductance (derived from the ratio of measured current jump to the applied voltage) of phi29, SPP1 and T4 was determined to be 4.52±0.33 nS, 4.10±0.22 nS, and 3.03±0.37 nS, respectively (FIG. 9E-G). T3 conductance distribution appeared as two peaks: 2.65±0.31 nS and 3.90±0.38 nS (FIG. 9H). The conductance values correspond to the respective pore sizes of phi29, SPP1, T3 and T4 portal channels (FIG. 9A-D).

Under a scanning voltage (−50 mV to +50 mV; 2.2 mV/s), the phi29, SPP1, T4 and T3 portal channels all display a linear Current-Voltage (I-V) relationship without voltage gating (FIG. 2I-L). When 100 mV was applied, the phi29, T4 and T3 portal channels remained stable, but the SPP1 portal channel started to gate (data not shown). In addition, the SPP1 portal channel had a stronger tendency to close the gate under negative voltages compared to positive potentials (data not shown).

Three Step Gating of Phi29, SPP1, T4 and T3 Portal Channels

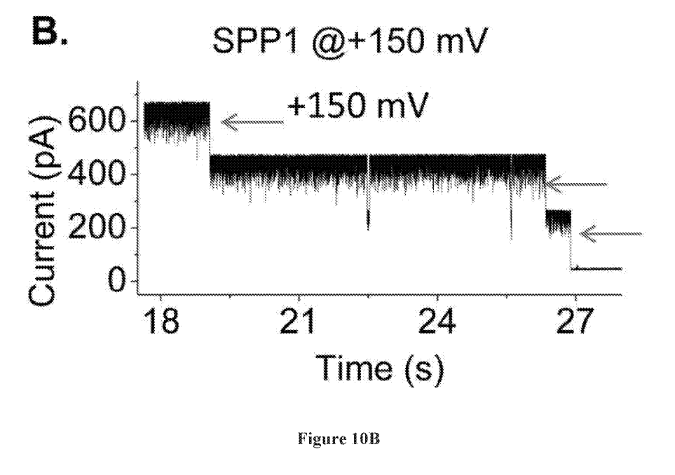

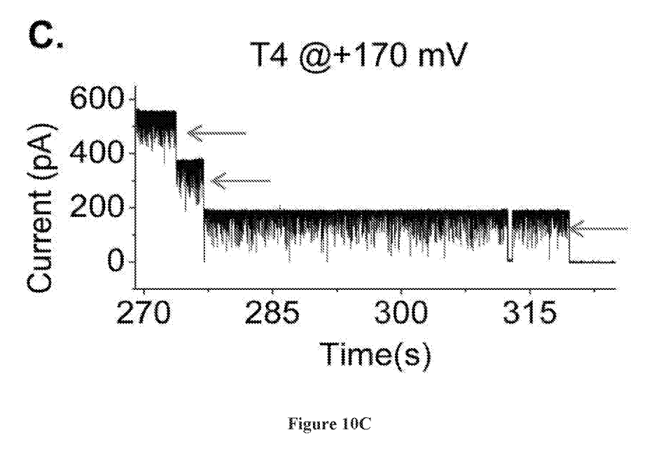

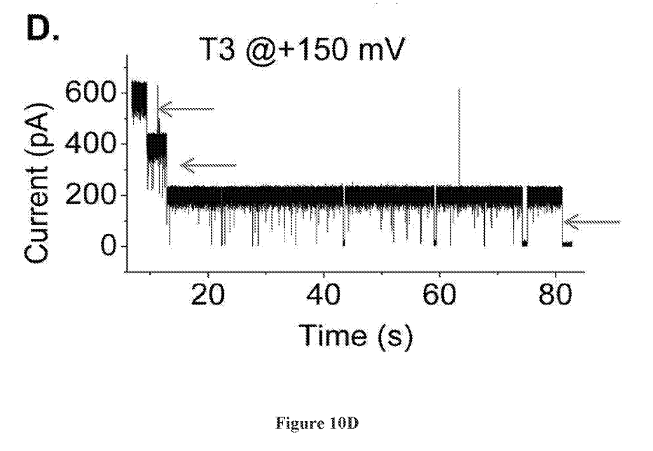

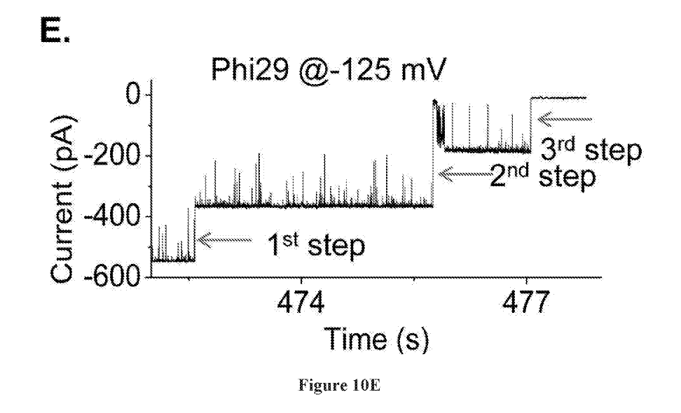

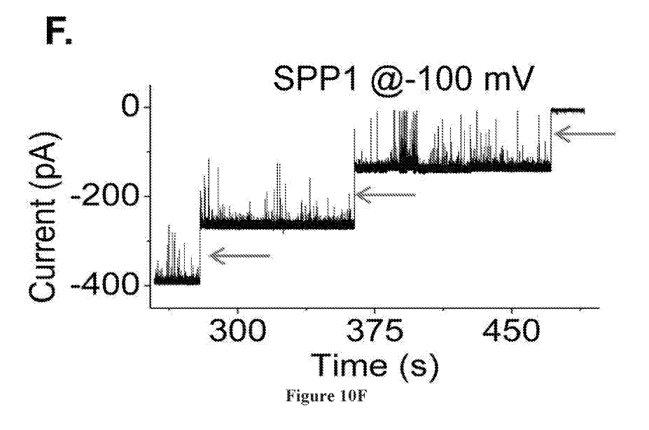

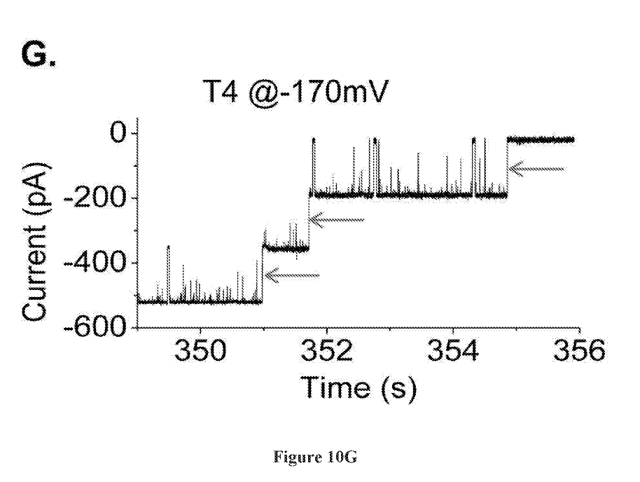

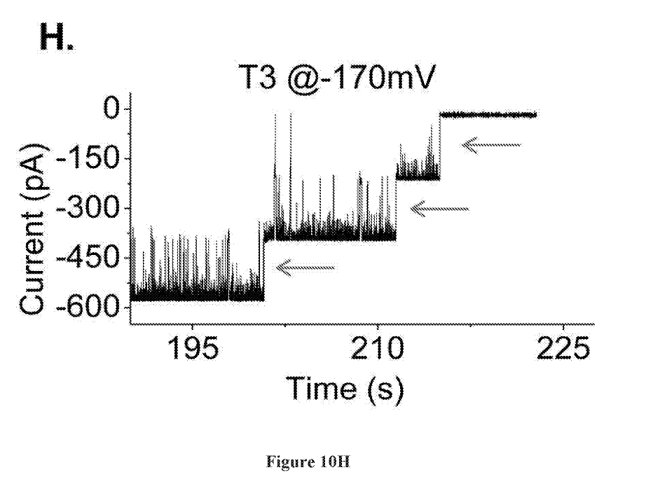

When a higher voltage (>100 mV) was applied, three distinct steps of conformational changes of the channel were observed in all four portal channels. Conformational changes were reflected by a reduction in electrical current of 33%, 66% and 99% for the first, second, and third step, respectively (FIG. 10A-11H). Three discrete step gating of the phi29, SPP1, T4 and T3 portal channels were observed under an applied positive voltage of +150 mV, +150 mV, +170 mV and +150 mV, respectively (FIG. 10A-D). Similar phenomena were observed under negative voltages of −125 mV, −100 mV, −175 mV and −125 mV for the four portals, respectively (FIG. 10E-H). These are the minimum voltages required for channel gating.

Discussion

The polymorphism of portal complexes assembled from overexpressed genes of bacterial viruses has been reported for many years. Although it is believed that the T4 and SPP1 portals exist as dodecamers in their biologically active state, the stoichiometry of the overexpressed portal gene products in different bacterial viruses has been reported to vary from 11-mer to 14-mer (Cingolani et al., 2002; Dube et al., 1993; Trus et al., 2004; Camacho et al., 2003; Tsuprun et al., 1994). Several studies revealed that the T3 portal structure is a mixed population of 12 and 13 subunits (Valpuesta et al., 2000). The diverse distribution of conductance for phi29, SPP1, T4, and T3 portals might represent various oligomeric states in these portal complexes. This is reflected by the two major peaks observed in the T3 conductance distribution (FIG. 9H).

It has been shown that all portal channels of dsDNA bacterial viruses display a left-handed channel wall configuration to facilitate the one-way traffic of dsDNA into procapsid by a revolution mechanism without rotation (Jing et al., 2010; Zhao et al., 2013; Schwartz et al., 2013; De-Donatis et al., 2014). The one-way valve mechanism is consistent with the findings of genome gating in SPP1, albeit gating mechanism proposed by these authors is based on the analysis of the channel structure after the completion of DNA packaging instead of during translocation (Chaban et al., 2015). The finding of the “push through a one-way valve” mechanism (Guo et al., 2016; Zhang et al., 2012) raises the question of how dsDNA is ejected during infection if the channel only permits dsDNA to translocate in one direction. We believe that during dsDNA translocation, the interaction of the dsDNA with the channel wall and the procapsid component next to the portal will trigger conformational changes of the portal. Therefore, the left-handed portal channel, which facilitates dsDNA advancement in one direction, will transition to a neutral or right-handed configuration in three steps to facilitate DNA ejection after DNA packaging is complete (De-Donatis et al., 2014).

Such conformational changes of portal proteins, as proposed above for ejection of the packaged dsDNA, have previously been proposed (Tang et al., 2008; Geng et al., 2011; Guo et al., 2005). Portal gate closing has been reported in SPP1 (Orlova et al., 2003) and speculated in T4 (Sun et al., 2015). Moreover, it was reported that SPP1 portal undergoes a concerted reorganization of the structural elements of its central channel during interaction with DNA. Structural rearrangements and gate closing were reported to associate with protein-protein and protein-DNA interactions, and a diaphragm-like mechanism for channel reduction and gate closing has been proposed (Chaban et al., 2015). The changes with discrete steps might be considered as the analogy of a camera lens by suggesting discrete f-stops, like f4.5, f8, f16, f32. However, the diaphragm proposal is difficult to reconcile with the data implying a right-handed twisting of the connector structure while comparing the free connector with the structure of the connector in the DNA-filled virion (Tang et al., 2008). The finding of the common discrete 3-step conformational change in T3, T4, SPP1 and phi29 implies a universal property of all portals. It is possible that the three gating steps may also correspond to the quantized steps of partial genome ejection observed in T3 (Serwer et al., 2014), and the partial packaging intermediates observed in phi29 (Bjornsti et al., 1983).

Conclusions

The motor channel of T3, SPP1, T4, and phi29 all display three discrete steps of voltage gating resulting from channel conformational changes. The result suggests that the one way inbound channel during the DNA packaging process is transformed into an outbound channel prepared for DNA ejection during the host infection.

### APPENDIX A

The following sequence listings correspond to the brief description of sequence listings set forth in paragraphs 0058-0061 above.

### SEQUENCE LISTING

### T3 Mutant Connector Sequence

Underlined is the site where mutagenesis is made.

SEQ ID NO. 1: DNA sequence
GTAACCTGCATATGGCTGATTCAAAACGTACAGGATTGGGCGAAGACGGTGCTAAAGCTAC
CTATGACCGCCTAACAAACGACCGTAGAGCCTATGAGACTCGTGCGGAGAACTGTGCGCAA
TACACCATTCCGTCCTTGTTCCCGAAGGAGTCCGATAACGAATCTACCGACTACACGACTCC
GTGGCAGGCTGTAGGTGCGCGGGGTCTCAACAATCTAGCCTCTAAGTTAATGCTTGCGTTAT
TCCCGATGCAGTCGTGGATGAAGCTGACCATTAGCGAATATGAGGCGAAGCAGCTTGTTGG
AGACCCTGATGGACTCGCTAAGGTGGACGAAGGTCTGTCAATGGTTGAGCGCATAATCATG
AACTATATCGAATCCAACAGTTACCGCGTAACACTCTTTGAGTGCCTCAAGCAGTTGATCGT
GGCTGGTAACGCCCTGCTTTACTTACCGGAACCAGAAGGTAGCTACAATCCGATGAAGCTGT
ACCGATTGTCTTCTTATGTTGTCCAAAGAGACGCATACGGCAATGTGTTACAGATTGTCACTC
GTGACCAGATAGCCTTTGGTGCTCTCCCGGAAGACGTTAGGTCTGCGGTAGAGAAATCTGGT
GGTGAGAAGAAGATGGACGAAATGGTCGATGTGTACACCCATGTGTATCTCGATGAAGAGT
CCGGCGATTACCTCAAGTACGAGGAAGTAGAGGACGTTGAGATTGATGGTTCCGATGCCAC
CTATCCGACTGACGCGATGCCCTACATTCCGGTTCGCATGGTTCGCATTGATGGCGAGTCTTA
CGGTCGCTCCTACTGTGAAGAATACTTAGGTGACTTAAGGTCGCTTGAGAATCTCCAAGAGG
CTATCGTTAAGATGAGTATGATTAGCGCGAAGGTCATTGGTCTGGTGAACCCGGCTGGCATT
ACGCAGCCCCGTAGATTAACCAAAGCTCAGACTGGTGACTTCGTTCCAGGCCGTCGAGAAG
ATATTGACTTCCTGCAACTGGAGAAGCAAGCTGACTTTACCGTAGCGAAAGCTGTGAGTGAC
CAGATAGAAGCACGCTTATCGTATGCCTTTATGTTGAACTCTGCGGTACAGCGCACAGGCGA
ACGTGTGACCGCCGAAGAGATTCGATACGTTGCGTCAGAACTGGAAGATACGCTTGGTGGC
GTCTACTCGATTCTGTCTCAAGAATTGCAATTGCCTCTGGTACGTGTGCTCTTGAAGCAACTC
CAAGCAACCTCGCAGATTCCTGAGCTACCGAAAGAAGCCGTTGAGCCTACTATCAGTACAG
GTCTGGAAGCAATTGGTCGTGGTCAAGACCTCGATAAGCTGGAGCGTTGCATCTCAGCGTGG
GCGGCTCTTGCCCCTATGCAGGGAGACCCGGACATTAATCTTGCTGTCATTAAGCTACGCAT
TGCTAACGCTATAGGTATTGATACTTCTGGTATCCTACTGACGGATGAACAGAAGCAAGCCC
TTATGATGCAGGATGCGGCACAAACAGGCGTCGAGAATGCTGCGGCTGCTGGTGGTGCTGG
TGTTGGTGCTTTGGCTACCTCAAGTCCAGAAGCCATGCAAGGTGCTGCTGCCAAGGCTGGCC
TCAACGCCACCGGTGGCCACCATCACCATCACCATTAG
SEQ ID NO. 2: Protein Sequence
Met A D S K R T G L G E D G A K A T Y D R L T N D R R A Y E T R
A E N C A Q Y T I P S L F P K E S D N E S T D Y T T P W Q A V G
A R G L N N L A S K L Met L A L F P Met Q S W Met K L T I S E Y
E A K Q L V G D P D G L A K V D E G L S Met V E R I I Met N Y I
E S N S Y R V T L F E C L K Q L I V A G N A L L Y L P E P E G S
Y N P Met K L Y R L S S Y V V Q R D A Y G N V L Q I V T R D Q I
A F G A L P E D V R S A V E K S G G E K K Met D E Met V D V Y T
H V Y L D E E S G D Y L K Y E E V E D V E I D G S D A T Y P T D
A Met P Y I P V R Met V R I D G E S Y G R S Y C E E Y L G D L R
S L E N L Q E A I V K Met S Met I S A K V I G L V N P A G I T Q
P R R L T K A Q T G D F V P G R R E D I D F L Q L E K Q A D F T
V A K A V S D Q I E A R L S Y A F Met L N S A V Q R T G E R V T
A E E I R Y V A S E L E D T L G G V Y S I L S Q E L Q L P L V R
V L L K Q L Q A T S Q I P E L P K E A V E P T I S T G L E A I G
R G Q D L D K L E R C I S A W A A L A P Met Q G D P D I N L A V
I K L R I A N A I G I D T S G I L L T D E Q K Q A L Met Met Q D
A A Q T G V E N A A A A G G A G V G A L A T S S P E A Met Q G A
A A K A G L N A T G G H H H H H H
T4 GP-20 MUTANT CONNECTOR SEQUENCE
UNDERLINED IS THE SITE WHERE MUTAGENESIS IS MADE.
SEQ ID NO. 3: DNA SEQUENCE
ATGAAATTTAATGTATTAAGTTTGTTTGCTCCATGGGCTAAAATGGACGAACGAAATTTTA
AAGACCAAGAAAAAGAAGATCTTGTTTCCATTACAGCCCCAAAGCTTGATGATGGAGCAA
GAGAATTTGAAGTAAGCTCGAATGAAGCTGCTTCTCCTTATAATGCTGCATTCCAAACAAT
TTTTGGTTCATATGAACCAGGAATGAAAACTACTCGTGAGCTTATTGATACATATCGTAAT
CTCATGAATAACTATGAAGTAGATAATGCAGTTTCAGAAATCGTTTCAGATGCTATCGTCT
ATGAAGATGATACTGAAGTCGTAGCGTTAAATTTGGATAAATCTAAATTTAGCCCAAAAA
TTAAAAATATGATGTTAGATGAATTTAGTGATGTATTAAATCATCTATCGTTTCAACGAAA
AGGTTCTGATCATTTTAGACGTTGGTATGTTGATTCAAGAATTTTCTTTCATAAAATCATTG
ATCCAAAACGTCCAAAAGAAGGCATAAAAGAATTACGTAGATTAGACCCTCGCCAAGTTC
AGTATGTTCGTGAAATTATAACAGAAACTGAAGCTGGCACAAAAATAGTTAAAGGTTACA
AAGAATATTTTATATATGATACTGCCCATGAGTCATATGCATGTGATGGTAGAATGTATGA
AGCTGGCACAAAAATAAAAATTCCTAAAGCTGCCGTCGTTTATGCCCATTCTGGATTAGTC
GATTGTTGCGGTAAAAATATCATCGGGTATTTGCATCGTGCTGTTAAACCTGCTAACCAAT
TAAAATTATTAGAAGATGCTGTAGTCATTTATCGCATTACTCGTGCTCCTGACCGTCGTGT
TTGGTATGTAGACACAGGTAATATGCCTGCTCGTAAAGCTGCTGAGCACATGCAACATGTT
ATGAACACGATGAAAAACCGTGTAGTATATGATGCATCAACAGGTAAAATAAAAAATCA
ACAGCATAATATGTCTATGACCGAAGACTATTGGTTGCAGCGCCGTGATGGTAAAGCTGT
GACAGAAGTTGATACTCTTCCTGGTGCTGATAATACTGGCAATATGGAAGATATTCGTTGG
TTTAGACAAGCTCTTTATATGGCATTACGTGTTCCTCTTTCACGCATTCCGCAAGACCAAC
AAGGCGGTGTGATGTTTGATTCTGGAACTAGCATTACACGTGATGAATTAACGTTTGCTAA
ATTTATTCGTGAGTTACAGCACAAGTTTGAAGAAGTTTTCCTAGATCCGCTTAAAACAAAT
CTTTTGCTTAAAGGTATAATCACAGAAGATGAGTGGAATGATGAAATAAATAATATTAAG
ATAGAATTTCATCGGGATAGCTACTTTGCTGAGCTCAAAGAAGCA
GAAATTTTGGAACGAAGAATTAATATGCTAACCATGGCAGAACCATTTATTGGTAAATAT
ATTTCTCACAGAACTGCTATGAAAGACATTTTGCAGATGACTGATGAAGAAATAGAACAA
GAAGCCAAGCAAATTGAAGAAGAGTCTAAAGAGGCTCGTTTCCAAGACCCCGACCAAGA
ACAAGAGGATTTTGGTGGCCACCATCACCATCACCATTAG
SEQ ID NO. 4: Protein Sequence
Met K F N V L S L F A P W A K Met D E R N F K D Q E K E D L V S
I T A P K L D D G A R E F E V S S N E A A S P Y N A A F Q T I F
G S Y E P G Met K T T R E L I D T Y R N L Met N N Y E V D N A V
S E I V S D A I V Y E D D T E V V A L N L D K S K F S P K I K N
Met Met L D E F S D V L N H L S F Q R K G S D H F R R W Y V D S
R I F F H K I I D P K R P K E G I K E L R R L D P R Q V Q Y V R
E I I T E T E A G T K I V K G Y K E Y F I Y D T A H E S Y A C D
G R Met Y E A G T K I K I P K A A V V Y A H S G L V D C C G K N
I I G Y L H R A V K P A N Q L K L L E D A V V I Y R I T R A P D
R R V W Y V D T G N Met P A R K A A E H Met Q H V Met N T Met K
N R V V Y D A S T G K I K N Q Q H N Met S Met T E D Y W L Q R R
D G K A V T E V D T L P G A D N T G N Met E D I R W F R Q A L Y
Met A L R V P L S R I P Q D Q Q G G V Met F D S G T S I T R D E
L T F A K F I R E L Q H K F E E V F L D P L K T N L L L K G I I
T E D E W N D E I N N I K I E F H R D S Y F A E L K E A E I L E
R R I N Met L T Met A E P F I G K Y I S H R T A Met K D I L Q Met
T D E E I E Q E A K Q I E E E S K E A R F Q D P D Q E Q E D F G
G H H H H H H

### APPENDIX B

### Reference Numbers in this Appendix B Correspond to Reference Numbers Set Forth in Section 1 (and FIGS. 1-8, 12-19)

1. Guo, P., I. Grainge, Z. Zhao, and M. Vieweger. 2014. Two classes of nucleic acid translocation motors: rotation and revolution without rotation. Cell Biosci. 4: 54 PM:25276341.
2. Hiroyuki Noji, and Masasuke Yoshida. 2001. The Rotary Machine in the Cell, ATP Synthase. J Biol Chem 276(3): 1665-1668.
3. Grainge, I. 2010. FtsK—a bacterial cell division checkpoint? Mol. Microbiol. 78: 1055-1057 PM:21155139.
4. Grainge, I. 2008. Sporulation: SpoIIIE is the key to cell differentiation. Curr. Biol. 18: R871-R872.
5. Aksyuk, A. A., P. G. Leiman, L. P. Kurochkina, M. M. Shneider, V. A. Kostyuchenko, V. V. Mesyanzhinov, and M. G. Rossmann. 2009. The tail sheath structure of bacteriophage T4: a molecular machine for infecting bacteria. EMBO J 28: 821-829 PM:19229296.
6. Chung, W. J., J. W. Oh, K. Kwak, B. Y. Lee, J. Meyer, E. Wang, A. Hexemer, and S. W. Lee. 2011. Biomimetic self-templating supramolecular structures. Nature 478: 364-368 PM:22012394.
7. Earnshaw, W. C., and S. R. Casjens. 1980. DNA packaging by the double-stranded DNA bacteriophages. Cell 21: 319-331.
8. Guo, P., C. Zhang, C. Chen, M. Trottier, and K. Garver. 1998. Inter-RNA interaction of phage phi29 pRNA to form a hexameric complex for viral DNA transportation. Mol. Cell. 2: 149-155.
9. Hendrix, R. W. 1998. Bacteriophage DNA packaging: RNA gears in a DNA transport machine (Minireview). Cell 94: 147-150.
10. Fang, H., P. Jing, F. Hague, and P. Guo. 2012. Role of channel Lysines and “Push Through a One-way Valve” Mechanism of Viral DNA packaging Motor. Biophysical Journal 102: 127-135.
11. Jing, P., F. Hague, D. Shu, C. Montemagno, and P. Guo. 2010. One-Way Traffic of a Viral Motor Channel for Double-Stranded DNA Translocation. Nano Lett. 10: 36203627.
12. Guo, P. 2014. Biophysical Studies Reveal New Evidence for One-Way Revolution Mechanism of Bacteriophage phi29 DNA Packaging Motor. Biophysical Journal 106: 1837-1838 PM:24806913.
13. Liu, S., G. Chistol, C. L. Hetherington, S. Tafoya, K. Aathavan, J. Schnitzbauer, S. Grimes, P. J. Jardine, and C. Bustamante. 2014. A viral packaging motor varies its DNA rotation and step size to preserve subunit coordination as the capsid fills. Cell 157: 702713 PM:24766813.
14. Ray, K., C. R. Sabanayagam, J. R. Lakowicz, and L. W. Black. 2010. DNA crunching by a viral packaging motor: Compression of a procapsid-portal stalled Y-DNA substrate. Virology 398: 224-232.
15. Dixit, A. B., K. Ray, and L. W. Black. 2012. Compression of the DNA substrate by a viral packaging motor is supported by removal of intercalating dye during translocation. Proc. Natl. Acad. Sci. U. S. A 109: 20419-20424 PM:23185020.
16. Serwer, P., E. T. Wright, Z. Liu, and W. Jiang. 2014. Length quantization of DNA partially expelled from heads of a bacteriophage T3 mutant. Virology 456-457: 157-170 PM:24889235.
17. Bjornsti, M. A., B. E. Reilly, and D. L. Anderson. 1983. Morphogenesis of bacteriophage □29 of Bacillus subtilis: oriented and quantized in vitro packaging of DNA protein gp3. J. Virol. 45: 383-396.
18. Urbaneja, M. A., S. Rivqas, J. L. Carrascosa, and J. M. Valpuesta. 1994. An intrinsic-tryptophan-fluorescence study of phage phi29 connector/nucleic acid interactions. Eur. J. Biochem. 225: 747-753.
19. Anon C. Tolley, and Nicola J. Stonehouse. 2008. Conformational changes in the connector protein complex of the bacteriophage phi29 DNA packaging motor. Computational and Mathematical Methods in Medicine 9: 327-337.
20. Tang, J. H., N. Olson, P. J. Jardine, S. Girimes, D. L. Anderson, and T. S. Baker. 2008. DNA poised for release in bacteriophage phi29. Structure 16: 935-943 ISI:000256816200014.
22. Hague, F., J. Li, H.-C. Wu, X.-J. Liang, and P. Guo. 2013. Solid-state and biological nanopore for real-time sensing of single chemical and sequencing of DNA. Nano Today 8: 56-74.
23. Branton, D., D. W. Deamer, A. Marziali, H. Bayley, S. A. Benner, T. Butler, V. M. Di, S. Garaj, A. Hibbs, X. Huang, S. B. Jovanovich, P. S. Krstic, S. Lindsay, X. S. Ling, C. H. Mastrangelo, A. Meller, J. S. Oliver, Y. V. Pershin, J. M. Ramsey, R. Riehn, G. V. Soni, V. Tabard-Cossa, M. Wanunu, M. Wiggin, and J. A. Schloss. 2008. The potential and challenges of nanopore sequencing. Nat. Biotechnol. 26: 1146-1153 PM:18846088.
24. Venkatesan, B. M., and R. Bashir. 2011. Nanopore sensors for nucleic acid analysis. Nature Nanotechnology 6: 615-624.
25. Healy, K. 2007. Nanopore-based single-molecule DNA analysis. Nanomedicine 2: 459-481 ISI:000249070400009.
26. Majd, S., E. C. Yusko, Y. N. Billeh, M. X. Macrae, J. Yang, and M. Mayer. 2010. Applications of biological pores in nanomedicine, sensing, and nanoelectronics. Current Opinion in Biotechnology 21: 439-476 ISI:000282717000007.
27. Kasianowicz, J. J., J. W. Robertson, E. R. Chan, J. E. Reiner, and V. M. Stanford. 2008. Nanoscopic porous sensors. Annu. Rev. Anal. Chem. (Palo. Alto. Calif.) 1: 737-766 PM:20636096.
28. Howorka, S., and Z. Siwy. 2009. Nanopore analytics: sensing of single molecules. Chem. Soc. Rev. 38: 2360-2384 PM:19623355.
29. Reiner, J. E., A. Balijepalli, J. W. Robertson, J. Campbell, J. Suehle, and J. J. Kasianowicz. 2012. Disease detection and management via single nanopore-based sensors. Chem. Rev. 112: 6431-6451 PM:23157510.
30. Wendell, D., P. Jing, J. Geng, V. Subramaniam, T. J. Lee, C. Montemagno, and P. Guo. 2009. Translocation of double-stranded DNA through membrane-adapted phi29 motor protein nanopores. Nat. Nanotechnol. 4: 765-772.
31. Jing, P., F. Hague, A. Vonderheide, C. Montemagno, and P. Guo. 2010. Robust Properties of Membrane-Embedded Connector Channel of Bacterial Virus Phi29 DNA Packaging Motor. Mol. Biosyst. 6: 1844-1852.
32. Wang, S., F. Hague, P. G. Rychahou, B. M. Evers, and P. Guo. 2013. Engineered Nanopore of Phi29 DNA-Packaging Motor for Real-Time Detection of Single Colon Cancer Specific Antibody in Serum. ACS Nano 7: 9814-9822 PM:24152066.
33. Hague, F., J. Lunn, H. Fang, D. Smithrud, and P. Guo. 2012. Real-Time Sensing and Discrimination of Single Chemicals Using the Channel of Phi29 DNA Packaging Nanomotor. ACS Nano 6: 3251-3261 PM:22458779.
34. Hague, F., S. Wang, C. Stites, L. Chen, C. Wang, and P. Guo. 2015. Single pore translocation of folded, double-stranded, and tetra-stranded DNA through channel of bacteriophage Phi29 DNA packaging motor. Biomaterials 53: 744-752.
35. Geng, J., S. Wang, H. Fang, and P. Guo. 2013. Channel size conversion of Phi29 DNA-packaging nanomotor for discrimination of single- and double-stranded nucleic acids. ACS Nano 7: 3315-3323 PM:23488809.
36. Geng, J., H. Fang, F. Hague, L. Zhang, and P. Guo. 2011. Three reversible and controllable discrete steps of channel gating of a viral DNA packaging motor. Biomaterials 32: 8234-8242.
37. Hague, F., and P. Guo. 2011. Membrane-embedded Channel of Bacteriophage Phi29 DNA-Packaging Motor for Translocation and Sensing of Double-stranded DNA. In Nanopores, Sensing and Fundamental Biological Interactions. S. M. Iqbal, and R. Bashir, editors. Springer, 77-106.
38. Hague, F., J. Geng, C. Montemagno, and P. Guo. 2013. Incorporation of Viral DNA Packaging Motor Channel in Lipid Bilayers for Real-Time, Single-Molecule Sensing of Chemicals and Double-Stranded DNA. Nat. Protoc. 8: 373-392.
39. Quinten, T. A., and A. Kuhn. 2012. Membrane interaction of the portal protein gp20 of bacteriophage T4. J Virol. 86: 11107-11114 PM:22855489.
40. Sun, S., V. B. Rao, and M. G. Rossmann. 2010. Genome packaging in viruses. Curr. Opin. Struct. Biol. 20: 114-120.
41. Guasch, A., J. Pous, B. Ibarra, F. X. Gomis-Ruth, J. M. Valpuesta, N. Sousa, J. L. Carrascosa, and M. Coll. 2002. Detailed architecture of a DNA translocating machine: the high-resolution structure of the bacteriophage phi29 connector particle. J. Mol. Biol. 315: 663-676.
42. Lebedev, A. A., M. H. Krause, A. L. Isidro, A. A. Vagin, E. V. Orlova, J. Turner, E. J. Dodson, P. Tavares, and A. A. Antson. 2007. Structural framework for DNA translocation via the viral portal protein. EMBO J. 26: 1984-1994.
43. Lhuillier, S., M. Gallopin, B. Gilquin, S. Brasiles, N. Lancelot, G. Letellier, M. Gilles, G. Dethan, E. V. Orlova, J. Couprie, P. Tavares, and S. Zinn-Justin. 2009. Structure of bacteriophage SPP1 head-to-tail connection reveals mechanism for viral DNA gating. Proc. Natl. Acad. Sci. U. S. A 106: 8507-8512 PM:19433794.
43. Driedonks, R. A., A. Engel, B. tenHeggeler, and R. van Driel. 1981. Gene 20 Product of Bacteriophage T4: Its Purification and Structure. J Mol Biol 152: 641-662.
44. Valpuesta, J. M., H. Fujisawa, S. Marco, J. M. Carazo, and J. Carrascosa. 1992. Three-dimensional structure of T3 connector purified from overexpressing bacteria. J Mol Biol 224: 103-112.
45. Carazo, J. M., H. Fujisawa, S. Nakasu, and J. L. Carrascosa. 1986. Bacteriophage T3 gene 8 product oligomer structure. Journal of ultrastructure and molecular structure research 94: 105-113.
46. Valpuesta, J. M., N. Sousa, I. barthelemy, J. J. Fernandez, H. Fujisawa, B. Ibarra, and J. L. Carrascosa. 2000. Structural analysis of the bacteriophage T3 head-to-tail connector. J. Struct. Biol. 131: 146-155.
47. Zhao, Z., E. Khisamutdinov, C. Schwartz, and P. Guo. 2013. Mechanism of one-way traffic of hexameric phi29 DNA packaging motor with four electropositive relaying layers facilitating anti-parallel revolution. ACS Nano 7: 4082-4092.
48. Schwartz, C., G. M. De Donatis, H. Zhang, H. Fang, and P. Guo. 2013. Revolution rather than rotation of AAA+ hexameric phi29 nanomotor for viral dsDNA packaging without coiling. Virology 443: 28-39.
49. De-Donatis, G., Z. Zhao, S. Wang, P. L. Huang, C. Schwartz, V. O. Tsodikov, H. Zhang, F. Hague, and P. Guo. 2014. Finding of widespread viral and bacterial revolution dsDNA translocation motors distinct from rotation motors by channel chirality and size. Cell Biosci 4: 30.
50. Guo, P., Z. Zhao, J. Haak, S. Wang, D. Wu, B. Meng, and T. Weitao. 2014. Common Mechanisms of DNA translocation motors in Bacteria and Viruses Using One-way Revolution Mechanism without Rotation. Biotechnology Advances 32: 853-872.
51. Kemp, P., M. Gupta, and I. J. Molineux. 2004. Bacteriophage T7 DNA ejection into cells is initiated by an enzyme-like mechanism. Mol. Microbiol. 53: 1251-1265 PM:15306026.
52. Hu, B., W. Margolin, I. J. Molineux, and J. Liu. 2013. The bacteriophage t7 virion undergoes extensive structural remodeling during infection. Science 339: 576-579 PM:23306440.
53. Yu, J., W. K. Leung, M. P. Ebert, E. K. Ng, M. Y. Go, H. B. Wang, S. C. Chung, P. Malfertheiner, and J. J. Sung. 2002. Increased expression of survivin in gastric cancer patients and in first degree relatives. Br. J. Cancer 87: 91-97 PM:12085263.
54. Zheng, H., A. S. Olia, M. Gonen, S. Andrews, G. Cingolani, and T. Gonen. 2008. A Conformational Switch in Bacteriophage P22 Portal Protein Primes Genome Injection. Mol. Cell. 29: 376-383.
55. Olia, A. S., P. E. Prevelige, J. E. Johnson, and G. Cingolani. 2011. Three-dimensional structure of a viral genome-delivery portal vertex. Nat Struct Mol Biol 18: 597-603.
56. Schwartz, C., G. M. De Donatis, H. Fang, and P. Guo. 2013. The ATPase of the phi29 DNA-packaging motor is a member of the hexameric AAA+ superfamily. Virology 443: 20-27.
57. Guo, P., C. Schwartz, J. Haak, and Z. Zhao. 2013. Discovery of a new motion mechanism of biomotors similar to the earth revolving around the sun without rotation. Virology 446: 133-143.
58. Bo Lu, W. Jin, Q. Zhao, and Y. Dapeng. 2012. Conductance and DNA Translocation Current Blockage of Solid-State Nanopores. Chinese Physics Letters 5.
59. Butler, T. Z., M. Pavlenok, I. M. Derrington, M. Niederweis, and J. H. Gundlach. 2008. Single-molecule DNA detection with an engineered MspA protein nanopore. Proc. Natl. Acad. Sci. U. S. A 105: 20647-20652 PM:19098105.
60. Rao, V. B., and M. Feiss. 2008. The bacteriophage DNA packaging motor. Annu. Rev. Genet. 42: 647-681 PM:18687036.
61. Cingolani, G., S. D. Moore, P. E. Prevelige, Jr., and J. E. Johnson. 2002. Preliminary crystallographic analysis of the bacteriophage P22 portal protein. J. Struct. Biol. 139: 4654.
62. Dube, P., P. Tavares, R. Lurz, and H. M. van. 1993. The portal protein of bacteriophage SPP1: a DNA pump with 13-fold symmetry. EMBO J 12: 1303-1309 PM:8467790.
63. Kocsis, E., M. E. Cerritelli, B. L. Trus, N. Cheng, and A. C. Steven. 1995. Improved methods for determination of rotational symmetries in macromolecules. Ultramicroscopy 60: 219-228 PM:7502382.
64. Trus, B. L., N. Cheng, W. W. Newcomb, F. L. Homa, J. C. Brown, and A. C. Steven. 2004. Structure and polymorphism of the UL6 portal protein of herpes simplex virus type 1. J. Virol. 78: 12668-12671.
65. International Patent Application Publication No. WO2010/062697

### APPENDIX C

### References in this Appendix C Correspond to Reference Numbers Set Forth in Section 2 (and FIGS. 8-11)

Bjornsti, M. A., Reilly, B. E., Anderson, D. L., 1983. Morphogenesis of bacteriophage ϕ29 of Bacillus subtilis: oriented and quantized in vitro packaging of DNA protein gp3. Journal of Virology 45, 383-396.
Cal, Y., Xiao, F., Guo, P., 2008. The effect of N- or C-terminal alterations of the connector of bacteriophage phi29 DNA packaging motor on procapsid assembly, pRNA binding, and DNA packaging. Nanomedicine 4, 8-18.
Camacho, A. G., Gual, A., Lurz, R., Tavares, P., Alonso, J. C., 2003. Bacillus subtilis bacteriophage SPP1 DNA packaging motor requires terminase and portal proteins. J. Biol. Chem. 278, 23251-23259.
Chaban, Y., Lurz, R., Brasiles, S., Cornilleau, C., Karreman, M., Zinn-Justin, S., Tavares, P., Orlova, E. V., 2015. Structural rearrangements in the phage head-to-tail interface during assembly and infection. Proc. Natl. Acad. Sci. U. S A 112, 7009-7014.
Cingolani, G., Moore, S. D., Prevelige, P. E., Jr., Johnson, J. E., 2002. Preliminary crystallographic analysis of the bacteriophage P22 portal protein. J. Struct. Biol. 139, 46-54.
Coulter, W. H., 1953. U.S. Pat. No. 2,656,508. In.
De-Donatis, G., Zhao, Z., Wang, S., Huang, P. L., Schwartz, C., Tsodikov, V. O., Zhang, H., Hague, F., Guo, P., 2014. Finding of widespread viral and bacterial revolution dsDNA translocation motors distinct from rotation motors by channel chirality and size. Cell Biosci 4, 30.
Dixit, A. B., Ray, K., Black, L. W., 2012. Compression of the DNA substrate by a viral packaging motor is supported by removal of intercalating dye during translocation. Proc. Natl. Acad. Sci. U.S.A 109, 20419-20424.
Dube, P., Tavares, P., Lurz, R., van, H. M., 1993. The portal protein of bacteriophage SPP1: a DNA pump with 13-fold symmetry. EMBO J 12, 1303-1309.
Fang, H., Jing, P., Hague, F., Guo, P., 2012. Role of channel Lysines and “Push Through a One-way Valve” Mechanism of Viral DNA packaging Motor. Biophysical Journal 102, 127-135.
Geng, J., Wang, S., Fang, H., Guo, P., 2013. Channel size conversion of Phi29 DNA-packaging nanomotor for discrimination of single- and double-stranded nucleic acids. ACS Nano 7, 3315-3323.
Geng, J., Fang, H., Haque, F., Zhang, L., Guo, P., 2011. Three reversible and controllable discrete steps of channel gating of a viral DNA packaging motor. Biomaterials 32, 8234-8242.
Grimes, S., Ma, S., Gao, J., Atz, R., Jardine, P. J., 2011. Role of phi29 connector channel loops in late-stage DNA packaging. J. Mol. Biol. 410, 50-59.
Guasch, A., Pous, J., Ibarra, B., Gomis-Ruth, F. X., Valpuesta, J. M., Sousa, N., Carrascosa, J. L., Coll, M., 2002. Detailed architecture of a DNA translocating machine: the high-resolution structure of the bacteriophage phi29 connector particle. J. Mol. Biol. 315, 663-676.
Guo, P., 2014. Biophysical Studies Reveal New Evidence for One-Way Revolution Mechanism of Bacteriophage phi29 DNA Packaging Motor. Biophysical Journal 106, 1837-1838.
Guo, P., Noji, H., Yengo, C M., Zhao, Z., Grainge, I., 2016. Biological nanomotors with revolution, linear, or rotation motion mechanism. Microbiology and Molecular Biology Reviews 80, 161-186.
Guo, P., Peterson, C., Anderson, D., 1987. Prohead and DNA-gp3-dependent ATPase activity of the DNA packaging protein gp16 of bacteriophage phi29. Journal of Molecular Biology 197, 229-236.
Guo, Y., Blocker, F., Xiao, F., Guo, P., 2005. Construction and 3-D computer modeling of connector arrays with tetragonal to decagonal transition induced by pRNA of phi29 DNA-packaging motor. J. Nanosci. Nanotechnol. 5, 856-863.
Haque, F., Geng, J., Montemagno, C., Guo, P., 2013a. Incorporation of Viral DNA Packaging Motor Channel in Lipid Bilayers for Real-Time, Single-Molecule Sensing of Chemicals and Double-Stranded DNA. Nat. Protoc. 8, 373-392.
Haque, F., Li, J., Wu, H.-C., Liang, X.-J., Guo, P., 2013b. Solid-state and biological nanopore for real-time sensing of single chemical and sequencing of DNA. Nano Today 8, 56-74.
Haque, F., Lunn, J., Fang, H., Smithrud, D., Guo, P., 2012. Real-Time Sensing and Discrimination of Single Chemicals Using the Channel of Phi29 DNA Packaging Nanomotor. ACS Nano 6, 3251-3261.
Harvey, S. C., 2015. The scrunchworm hypothesis: Transitions between A-DNA and B-DNA provide the driving force for genome packaging in double-stranded DNA bacteriophages. Journal of Structural Biology 189, 1-8.
Hu, B., Margolin, W., Molineux, I. J., Liu, J., 2013. The bacteriophage t7 virion undergoes extensive structural remodeling during infection. Science 339, 576-579.
Jing, P., Hague, F., Shu, D., Montemagno, C., Guo, P., 2010. One-Way Traffic of a Viral Motor Channel for Double-Stranded DNA Translocation. Nano Lett. 10, 3620-3627.
Lebedev, A. A., Krause, M. H., Isidro, A. L., Vagin, A. A., Orlova, E. V., Turner, J., Dodson, E. J., Tavares, P., Antson, A. A., 2007. Structural framework for DNA translocation via the viral portal protein. EMBO Journal 26, 1984-1994.
Lhuillier, S., Gallopin, M., Gilquin, B., Brasiles, S., Lancelot, N., Letellier, G., Gilles, M., Dethan, G., Orlova, E. V., Couprie, J., Tavares, P., Zinn-Justin, S., 2009. Structure of bacteriophage SPP1 head-to-tail connection reveals mechanism for viral DNA gating. Proc. Natl. Acad. Sci. U.S.A 106, 8507-8512.
Orlova, E. V., Gowen, B., Droge, A., Stiege, A., Weise, F., Lurz, R., van, H. M., Tavares, P., 2003. Structure of a viral DNA gatekeeper at 10 A resolution by cryo-electron microscopy. EMBO Journal 22, 1255-1262.
Quinten, T. A., Kuhn, A., 2012. Membrane interaction of the portal protein gp20 of bacteriophage T4. J Virol. 86, 11107-11114.
Ray, K., Sabanayagam, C. R., Lakowicz, J. R., Black, L. W., 2010. DNA crunching by a viral packaging motor: Compression of a procapsid-portal stalled Y-DNA substrate. Virology 398, 224-232.
Schwartz, C., De Donatis, G. M., Zhang, H., Fang, H., Guo, P., 2013. Revolution rather than rotation of AAA+ hexameric phi29 nanomotor for viral dsDNA packaging without coiling. Virology 443, 28-39.
Serwer, P., Wright, E. T., Liu, Z., Jiang, W., 2014. Length quantization of DNA partially expelled from heads of a bacteriophage T3 mutant. Virology 456-457, 157-170.
Sun, L., Zhang, X., Gao, S., Rao, P. A., Padilla-Sanchez, V., Chen, Z., Sun, S., Xiang, Y., Subramaniam, S., Rao, V. B., Rossmann, M. G., 2015. Cryo-EM structure of the bacteriophage T4 portal protein assembly at near-atomic resolution. Nat. Commun. 6, 7548.
Tang, J. H., Olson, N., Jardine, P. J., Girimes, S., Anderson, D. L., Baker, T. S., 2008. DNA poised for release in bacteriophage phi29. Structure 16, 935-943.
Tolley, A. C., Stonehouse, N. J., 2008. Conformational changes in the connector protein complex of the bacteriophage phi29 DNA packaging motor. Computational and Mathematical Methods in Medicine 9, 327-337.
Trus, B. L., Cheng, N., Newcomb, W. W., Homa, F. L., Brown, J. C., Steven, A. C., 2004. Structure and polymorphism of the UL6 portal protein of herpes simplex virus type 1. Journal of Virology 78, 12668-12671.
Tsuprun, V., Anderson, D., Egelman, E. H., 1994. The Bacteriophage ϕ29 head-tail connector shows 13-Fold symmetry in both hexagonally packed arrays and as single particles. Biophys. J. 66, 2139-2150.
Urbaneja, M. A., Rivqas, S., Carrascosa, J. L., Valpuesta, J. M., 1994. An intrinsic-tryptophan-fluorescence study of phage phi29 connector/nucleic acid interactions. Eur. J. Biochem. 225, 747-753.
Valpuesta, J. M., Sousa, N., barthelemy, I., Fernandez, J. J., Fujisawa, H., Marra, B., Carrascosa, J. L., 2000. Structural analysis of the bacteriophage T3 head-to-tail connector. J. Struct. Biol. 131, 146-155.
Wang, S., Hague, F., Rychahou, P. G., Evers, B. M., Guo, P., 2013. Engineered Nanopore of Phi29 DNA-Packaging Motor for Real-Time Detection of Single Colon Cancer Specific Antibody in Serum. ACS Nano 7, 9814-9822.
Wendell, D., Jing, P., Geng, J., Subramaniam, V., Lee, T. J., Montemagno, C., Guo, P., 2009. Translocation of double-stranded DNA through membrane-adapted phi29 motor protein nanopores. Nat. Nanotechnol. 4, 765-772.
Zhang, H., Schwartz, C., De Donatis, G. M., Guo, P., 2012. “Push Through One-Way Valve” Mechanism of Viral DNA Packaging. Adv. Virus Res 83, 415-465.
Zhao, Z., Khisamutdinov, E., Schwartz, C., Guo, P., 2013. Mechanism of one-way traffic of hexameric phi29 DNA packaging motor with four electropositive relaying layers facilitating anti-parallel revolution. ACS Nano 7, 4082-4092.

### APPENDIX D

### This Appendix D Includes Additional Information Relating to FIGS. 8-11

FIG. 8: Structures of phi29, SPP1, and T4 portal channels. Top view, side view and single subunit of phi29 (A); SPP1 (B); and T4 (C) portal protein. Phi29 gp10 PDB: 1FOU; SPP1 gp6 PDB: 2JES; T4 gp20 PDB: 3JA7.
FIG. 9: Representative current traces showing insertion of phi29 (A), SPP1 (B), T4 (C) and T3 (D) portal channels into planar lipid membrane. Applied voltage: +50 mV. Histogram showing the conductance distribution of phi29 (E), SPP1 (F), T4 (G) and T3 (H) portal channels. Applied voltage: +50 mV. Current-Voltage trace under a ramping potential (−50 mV to +50 mV; 2.2 mV/s) for phi29 (single channel) (I), SPPI (two channels) (J), T4 (one channel) (K) and T3 (three channels) (L) portals. Conducting buffer: 1 M KCl, 5 mM HEPES, pH 7.8
FIG. 10: Three step gating associated with conformational changes of phi29 (A), SPP1 (B), T4 (C), and T3 (D) portal channel under positive trans-membrane voltages. Three steps gating associated with conformational changes of phi29 (E), SPP1 (F), T4 (G) and T3 (H) portal channels under negative trans-membrane voltages.
FIG. 11: Coomasie-blue stained 10% SDS-PAGE showing the molecular weight differences of single subunit of phi29 (36 kDa), SPP1 (56 kDa), T4 (60 kDa) and T3 (59 kDa) portal channels.

1. An engineered nucleic acid molecule encoding a T3 or T4 viral connector polypeptide variant, wherein the polypeptide comprises an amino acid sequence having at least 95% sequence identity to SEQ ID NO: 2, or SEQ ID NO: 4.

2. The nucleic acid molecule of claim 1, wherein the polypeptide comprises an amino acid sequence at least 99% sequence identity to SEQ ID NO: 2, or SEQ ID NO: 4.

3. The nucleic acid molecule of claim 1, wherein the polypeptide comprises the amino acid sequence of SEQ ID NO: 2, or SEQ ID NO:4.

4. The nucleic acid molecule of claim 1, comprising the sequence of SEQ ID NO: 1 or SEQ ID NO: 3.

5. An engineered T3 or T4 viral connector polypeptide variant, wherein the polypeptide comprises an amino acid sequence having at least 95% sequence identity to the sequence of SEQ ID NO: 2 or SEQ ID NO: 4.

6. The viral connector polypeptide variant of claim 5, wherein the polypeptide comprises an amino acid sequence having at least 99% sequence identity to the sequence of SEQ ID NO: 2 or SEQ ID NO: 4.

7. The viral connector polypeptide variant of claim 5, wherein the polypeptide comprises an amino acid sequence having the sequence of SEQ ID NO: 2 or SEQ ID NO: 4.

8. The viral connector polypeptide variant of claim 5, comprises the sequence of SEQ ID NO. 2 or SEQ ID NO. 4.

9. An artificial conductive channel-containing voltage-gated membrane complex, comprising:
A membrane layer; and
an isolated DNA-packaging motor connector protein that is incorporated into the membrane layer to form an aperture through which conductance can occur when an electrical potential is applied across the membrane, wherein the DNA-packaging motor connector protein comprises a homododecamer of viral DNA-packaging motor connecting protein polypeptide subunits, and wherein the subunits comprises an amino acid sequence having at least 95% identity to SEQ ID NO: 2 or SEQ ID NO: 4.

10. The membrane of claim 9, wherein the viral DNA-packaging motor connector protein polypeptide subunits comprises an amino acid sequence having at least 99% identity to SEQ ID NO: 2 or SEQ ID NO: 4.

11. The membrane of claim 9, wherein the viral DNA-packaging motor connector protein polypeptide subunits comprises SEQ ID NO: 2 or SEQ ID NO: 4.

12. The membrane of claim 9, wherein the viral DNA-packaging motor connector protein polypeptide subunits is encoded by a nucleic acid molecule comprising SEQ ID NO. 1 or SEQ ID NO. 3.

13. The membrane of claim 9, the subunit further comprising an affinity/alignment domain.

14. The membrane of claim 9, wherein the affinity/alignment domain comprises a polypeptide of
(i) a Strep-11 tag sequence WSHPQRFEK
(ii) a polyhistidine polypeptide tag of 3, 4, 5, 6, 7, 8, 9, 10, 11 or 12 contiguous histidine residues,
(iii) a polyarginine polypeptide of 3, 4, 5, 6, 7, 8, 9, 10, 11 or 12 contiguous arginine residues,
(iv) an HIV Tat polypeptide of sequence YGRKKRRQRR, and
(v) a peptide tag of sequence DRATPY.

15. The membrane of claim 9, wherein the membrane translocates double-stranded DNA through the aperture when the electrical potential is applied.

16. The membrane of claim 15, wherein the conductance occurs in the conductive channel-containing membrane with voltage-gating when electrical potential is applied.

17. The membrane of claim 15, wherein the applied electrical potential is greater than about 100 mV.

18. The membrane of claim 15, wherein the applied electrical potential is less than about −100 mV.

19. The membrane of claim 15, wherein the membrane layer comprises a lipid layer.

20. The membrane of claim 19, wherein the lipid layer comprises amphipathic lipids.

21. The membrane of claim 19, wherein the lipid layer is selected from the group consisting of a planar membrane layer and a liposome.

22. The membrane of claim 20, wherein the amphipathic lipids comprise phospholipids and the lipid layer comprises a lipid bilayer.

23. The membrane of claim 21, wherein the liposome is selected from the group consisting of a multilamellar liposome and a unilamellar liposome.

24. The membrane of claim 15, wherein the incorporated viral DNA-packaging motor connector protein is mobile in the membrane layer.

25. A method of sensing a molecule using a conductive channel-containing membrane of any one of claims 9-22, comprising
contacting the molecule with a conductive channel-containing membrane which comprises a membrane layer and incorporated therein one or a plurality of isolated viral DNA-packaging motor connector proteins,
applying an electrical potential,
detecting electrical current change, wherein the current change is a discrete a 3-step change.

26. The method of claim 25, wherein the discrete 3-step current change is about 33%, about 66%, and 99% reduction in each step.

27. The method of claim 25, wherein the electrical potential is greater than about 100 mV.

28. The method of claim 25, wherein the electrical potential is less than about −100 mV.

29. The method of claim 25, wherein the molecule is a polypeptide.

30. The method of claim 25, wherein the molecule is a nucleic acid molecule.

31. The method of claim 25, wherein the nucleic acid molecule is a double-stranded nucleic acid molecule.

32. A method of DNA sequencing using a conductive channel-containing membrane of any one of claims 9-22.
